(19) United States
(12) Patent Application Publication
(10) Pub. No.: US 2022/0257780 A1
(43) Pub. Date: Aug. 18, 2022

(54) MICRONIZED DRUG RESINATE-BASED PHARMACEUTICAL COMPOSITIONS AND METHODS OF PREPARATION THEREOF

(71) Applicant: Brillian Pharma Inc., Monmouth Junction, NJ (US)

(72) Inventor: Nuo Wang, Newtown, PA (US)

(73) Assignee: Brillian Pharma Inc., Monmouth Junction, NJ (US)

(21) Appl. No.: 17/618,020

(22) PCT Filed: Oct. 7, 2020
(86) PCT No.: PCT/US20/54517

§ 371 Date: Dec. 10, 2021

Related U.S. Application Data
(60) Provisional application No. 62/913,555, filed on Oct. 10, 2019.

(51) Int. Cl. A61K 47/58; A61K 9/16; A61K 45/06

(52) U.S. Cl. CPC A61K 47/585; A61K 9/1623; A61K 45/06; A61K 9/1641; A61K 9/1652; A61K 9/1635

(57) ABSTRACT

This disclosure provides a pharmaceutical composition for oral administration, which comprises micronized ion-exchange resin particles having particle sizes less than 50 pm and at least one therapeutic agent releasably bound to the micronized resin particles through ionic interaction to form resin-therapeutic agent complexes. The resin-therapeutic agent complexes have particle sizes less than 50 pm, and the pharmaceutical composition is formulated as a dosage form providing uniform dispersion of the resin-therapeutic agent complexes with a substantially masked taste of the therapeutic agent, and a reduced gritty mouthfeel for geriatric patients and pediatric patients.

### CROSS-REFERENCE TO RELATED APPLICATIONS

This application claims priority under 35 U.S.C. § 119(e) to U.S. Provisional Patent Application No. 62/913,555, filed Oct. 10, 2019. The foregoing applications are incorporated by reference herein.

### FIELD OF THE INVENTION

The present invention relates generally to pharmaceutical compositions containing drug resinate and more specifically to pharmaceutical compositions containing micronized drug resinate, method of preparation thereof, and methods of use thereof.

### BACKGROUND OF THE INVENTION

Orally administered drugs are provided to the patient in many dosage forms such as tablets, capsules, solutions, or suspensions. Many patients, including pediatric and geriatric patients, prefer a liquid oral dosage form to a solid dosage form. A liquid dosage is preferred by this class of patients because of the ease with which it may be swallowed. Additionally, patients may be more inclined to comply with their medication instructions if the dosages are easier to ingest. A common problem associated with liquid pharmaceutical dosage forms is the often disagreeable taste which may manifest itself when the drug is ingested in a liquid dosage form. It remains a challenge to develop formulations suitable for pediatric and geriatric patients as they generally have a low tolerance for disagreeable taste.

Pediatric patients are a special group of patients that is heterogeneous. The International Conference of Harmonization divides childhood into five age groups: preterm newborn infants; term newborn infants (0-27 days); infants and toddlers (1-23 months); children (2-11 years); and adolescents (12-16 years in the United States or 12-18 years in the European Union). Each group requires certain characters of drug formulation to be age-appropriate.

Child-friendly formulations generally have higher requirements in palatability, such as taste and mouthfeel. More than 90% of pediatricians in the US reported that a drug's taste and palatability were the greatest barriers to completing treatment. Tasteless or palatable drug formulation can minimize the loss of medication from spillage and/or spitting. Therefore, it is essential to taste mask or taste conceal an unpleasant drug to assure patient compliance and adherence. Studies by Lopez F L et al. and E. Imai et al. have also shown that when the number of particles in a fixed volume of medium increases, the perception of grittiness increases (Lopez F L et al., Effect of formulation variables on oral grittiness and preferences of multiparticulate formulations in adult volunteers, European Journal of Pharmaceutical Sciences 92 (2016) 156-162; Imai E et al., Degree of Grittiness Perceived as Mouth Feeling, J. Home Econ. Jpn. Vol. 46 No. 12; 1151-1158 (1995)). This means the higher concentration of particles in a suspension will result in reduced palatability.

Due to the heterogeneous nature of pediatric patients, dose adjustment according to the needs of the individual patient group is often required. To make matters worse, due to the lack of pediatric formulations, in the European Union, 45% to 60% of all medicines are given to children off-label. This trend is also true for 90% of medicines administered to neonates and infants, particularly in PICUs [European Medicines Agency. Report on the survey of all pediatric uses of medicinal products in Europe. Dec. 10, 2010, EMA/794083/2009]. In the United States, two-thirds of medicines used in pediatrics are off-label; worldwide, this proportion is up to three quarters (Ivanovska et al., Pediatrics August 2014, 134 (2) 361-372). Therefore, dosing flexibility is essential for pediatric drug formulations. Among the common dosage forms, liquid formulations provide the best dosing flexibility.

Size/amount of solid dosage forms (e.g., tablets, capsules, powder, pellets/beads) are also important for pediatric patients. For liquid formulations, EMA proposes that the maximum recommended single dosing volume is 5 ml for children aged below 4 years and 10 ml for children aged between 4 and 12 years. The smaller dosing size/amount or volume will require a higher degree of uniformity in the respective dosage forms. For example, mini-tablets 2-3 mm in diameter will require a much higher degree of uniformity of the compression blend than that for conventional bigger size tablets to assure content uniformity. Oral suspensions will require a much higher degree of uniform dispersion of particles in the suspending media and also dispersion stability to assure that each 5 or 10 ml dosing portion delivers consistent drug content.

Currently, only a small fraction of all marketed drugs are available in formulations that are age-appropriate. Resinate has been a drug delivery candidate for developing taste concealed age-appropriate drug formulations in recent years. Ion exchange resins or polymers are crosslinked, water-insoluble polymer matrix carrying ionizable functional groups. The resins can interact with a molecule carrying counter ions. Ion exchange can be defined as a reversible process in which ions of like sign are exchanged between liquid and solid, a highly insoluble body in contact with it. Such ion-exchange interaction is described by H. F. Walton in “Principles of Ion Exchange” (pp. 213-343). The high molecular weight created by cross-linking makes the resin water-insoluble and cannot be absorbed by the body. Such property makes the resin biologically pharmacologically inert and a good material as pharmaceutical excipients. Drugs with ionic groups can be loaded onto the resins by the same ion exchanging reaction and form a drug-resin complex (drug resinate). For example, U.S. Pat. No. 2,990,332 describes a pharmaceutical preparation for oral administration to a patient, comprising in a dosage unit form a therapeutically effective amount of cross-linked sulfonic acid cation exchange resin having a gastro-intestinal absorbable pharmaceutical organic drug containing a basic nitrogen group ionically bound to the resin to form an adsorption compound. WO1991013612A1 describes a composition for controlled and sustained release of a pharmaceutically acceptable drug comprising a drug-resin complex formed from an ion-exchange resin and a pharmacologically active drug. However, it did not describe the characteristics, such as palatability, of the described drug-resin complex.

Accordingly, there remains a strong need for developing age-appropriate formulations, such as formulations for pediatric and geriatric patients, with improved palatability and uniformity.

### SUMMARY OF THE INVENTION

This disclosure addresses the need mentioned above in a number of aspects. In one aspect, this disclosure provides a pharmaceutical composition for oral administration. The pharmaceutical composition comprises micronized ion-exchange resin particles having particle sizes less than 50 μm, and at least one therapeutic agent releasably bound to the micronized resin particles through ionic interaction to form resin-therapeutic agent complexes. The resin-therapeutic agent complexes have particle sizes less than 50 μm, and when the pharmaceutical composition is formulated as a dosage form. The dosage form can be a liquid dosage form or a solid dosage form.

The liquid dosage form comprises the resin-therapeutic agent complexes uniformly dispersed with less than 1 wt % of the micronized resin particles in the form of aggregates, thereby the taste of the therapeutic agent is substantially masked and the dosage form has a reduced gritty mouth feel compared to a dosage form containing resin particles with particle sizes larger than 50 μm.

When the pharmaceutical composition is formulated as a solid dosage form and when the solid dosage form is re-dispersed or reconstituted in a liquid medium (e.g., water, milk, or saliva during dwelling in oral cavity), the resin-therapeutic agent complexes can be uniformly dispersed with less than 1 wt % of the micronized resin particles in the form of aggregates, thereby the taste of the therapeutic agent is substantially masked and the dosage form has a reduced gritty mouth feel compared to a dosage form containing resin particles with particle sizes larger than 50 μm.

In some embodiments, the pharmaceutical composition further comprises a dispersing agent, thereby when the pharmaceutical composition is re-dispersed in a liquid medium, uniform dispersion of the resin-therapeutic agent complexes is produced and less than 1 wt % of the resin particles are in the form of aggregates.

The dispersing agent can be (1) a water-soluble substance selected from the group consisting of water-soluble polymers, hydrophilic surfactants, sugars, such as sodium alginate, gelatin, Arabic gum, agarose, polyvinyl pyrrolidone, hydroxypropyl methylcellulose, hydroxypropyl cellulose, sodium carboxymethyl cellulose, mannitol, lactose, sucrose, sodium lauryl sulfate, poloxamers, or combinations thereof, or (2) a water-insoluble but hydrophilic and swellable material commonly referred to as “disintegratant” selected from the group consisting of cross-linked or non-crosslinked synthetic or natural polymers, such as sodium starch glycolate, cross-linked polyvinylpyrrolidone, croscarmellose sodium, and alginic acid. For a pharmaceutical composition intended to be dispersed into an organic medium, the dispersing agent can be a solvent-soluble material selected from the group consisting of solvent-soluble polymers, lipophilic surfactants, phospholipids, fatty acids, such as polyvinyl pyrrolidone, phosphatidylcholine, phosphatidylethanolamine, stearate acid, oleic acid, or combinations thereof.

The dosage form can be one of suspension, dry powder for suspension, orally disintegrating tablets, mini-tablets with the longest dimension less than or equal to 3 mm, chewable tablets, oral jelly, and oral gummies.

In some embodiments, the dosage form is a dry powder for suspension, which comprises:
(a) between about 0.4% (w/w) and about 50% (w/w) (e.g., 0.4%, 0.6%, 0.8%, 1%, 4%, 8%, 12%, 16%, 20%, 24%, 28%, 32%, 36%, 40%, 44%, 48%, 50%) of the therapeutic agent;
(b) between about 0.4% (w/w) and about 99.6% (w/w) (e.g., 0.4%, 1%, 2%, 5%, 10%, 15%, 20%, 25%, 30%, 35%, 40%, 45%, 50%, 55%, 60%, 65%, 70%, 75%, 80%, 82%, 84%, 86%, 88%, 90%, 94%, 96%, 98%, 99.6%) of the micronized ion-exchange particles;
(c) between about 0% (w/w) and about 60% (w/w) of a dispersing aid or a dispersing agent;
(d) between about 0% (w/w) and about 40% (w/w) of a suspending agent or thickener;
(e) between about 0% (w/w) and about 20% (w/w) of an additional agent selected from the group consisting of a flavoring agent, a preservative, a pH adjusting agent, an antifoaming agent, a coloring agent, an antioxidant and a combination thereof.

In some embodiments, the particle size of the resin-therapeutic agent complex is from about 0.5 μm to about 40 μm, from about 1 μm to about 30 μm, or from about 1 μm to about 20 μm.

In some embodiments, the ratio of the therapeutic agent to the resin particle is in the range from 5:1 to 1:100 by weight, calculated on a moisture-free basis. In some embodiments, the ratio of the therapeutic agent to the resin particle is in the range from 2:1 to 1:20 by weight.

In some embodiments, the resin particle is an anionic ion-exchange resin particle or a cationic ion-exchange resin particle. The resin particle can be a cross-linked sulfonated polystyrene ion-exchange resin, a cross-linked methacrylic acid, and divinylbenzene copolymer ion-exchange resin, a cross-linked copolymer of diethylenetriamine and 1-chloro-2,3-epoxy propane ion-exchange resin, or a cross-linked copolymer of styrene and divinylbenzene with quaternary ammonium functionality ion-exchange resin. In some embodiments, the resin particle is Amberlite IRP-69, Amberlite IRP-64, Colestipol hydrochloride, or Duolite AP143/1093.

In some embodiments, the resin particle has an ion-exchange capacity of less than 6 milliequivalents per gram (meq/g) of dry resin.

In some embodiments, the pharmaceutical composition comprises from about 1 percent to about 90 percent by weight of the drug-resin particles.

In some embodiments, the therapeutic agent is acidic, including the therapeutic agent that contains a carboxyl group. In some embodiments, the therapeutic agent can be one of dehydrocholic acid, diflunisal, ethacrynic acid, fenoprofen, furosemide, gemfibrozil, ibuprofen, naproxen, phenytoin, probenecid, sulindac, theophylline, salicylic acid, and acetylsalicylic acid.

In some embodiments, wherein the therapeutic agent is basic, including the therapeutic agent contains an amine group. In some embodiments, the therapeutic agent can be one of acetophenazine, amitriptyline, amphetamine, benztropine, biperiden, bromodiphenhydramine, brompheniramine, carbinoxamine, chlorcyclizine, chlorpheniramine, chlorphenoxamine, chlorpromazine, clemastine, clomiphene, clonidine, codeine, cyclizine, cyclobenzaprine, cyproheptadine, desipramine, dexbrompheniramine, dexchlorpheniramine, dextroamphetamine, dextromethorphan, dicyclomine, diphemanil, diphenhydramine, doxepin, doxylamine, ergotamine, fluphenazine, haloperidol, hydrocodone, hydroxychloroquine, hydroxyzine, hyoscyamine, imipramine, levopropoxyphene, maprotiline, meclizine, mepenzolate, meperidine, mephentermine, mesoridazine, methadone, methdilazine, methscopolamine, methysergide, metoprolol, nortriptyline, no scapine, nylindrin, orphenadrine, papaverine, pentazocine, phendimetrazine, phentermine, phenylpropanolamine, pyrilamine, tripelennamine, triprolidine, promazine, propoxyphene, propanolol, pseudoephedrine, pyrilamine, quinidine, scopolamine, dextromethorphan, chlorpheniramine, and codeine.

In some embodiments, the therapeutic agent is amphoteric. In some embodiments, the therapeutic agent can be one of aminocaproic acid, amino salicylic acid, hydromorphone, isoxsuprine, levorphanol, melphalan, morphine, nalidixic acid, and para-amino salicylic acid.

In some embodiments, the therapeutic agent is selected from the group consisting of analeptic agents; analgesic agents; anesthetic agents; antiasthmatic agents; antiarthritic agents; anticancer agents; anticholinergic agents; anticonvulsant agents; antidepressant agents, antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism active agents; antipruritic agents; antipsychotic agents; antipyretic agents; antispasmodic agents; antitubercular agents; antiulcer agents; antiviral agents; anxiolytic agents; appetite Suppressants (anorexic agents); attention deficit disorder and attention deficit hyper activity disorder active agents; cardiovascular agents including calcium channel blockers and antianginal agents; central nervous system (CNS) agents; beta-blockers and antiar rhythmic agents; central nervous system stimulants; diuretics; genetic materials; hormonolytics; hypnotics; hypoglycemic agents; immuno suppressive agents; muscle relaxants; narcotic antagonists; nicotine; nutritional agents; parasym patholytics; peptide active agents; psychoStimulants; sedatives; Sialagogues, steroids; Smoking cessation agents; Sympathomimetics; tranquilizers; vasodilators; beta-agonist; tocolytic agents; and combinations thereof.

In another aspect, this disclosure also provides a method for preparing a pharmaceutical composition for oral administration. The method comprises: (1) micronizing ion-exchange resin particles by subjecting a suspended resin, either in air or a liquid medium, comprising ion-exchange resin particles having particle sizes larger than 50 μm to a size reduction process one or more times to obtain micronized ion-exchange resin particles having particle sizes equal to or less than 50 μm; (2) contacting the resulting micronized ion-exchange resin particles with at least one therapeutic agent to form resin-therapeutic agent complexes; and (3) admixing with a pharmaceutically acceptable carrier to form a pharmaceutical composition having uniform dispersion of the resin-therapeutic agent complexes and less than 1 wt % of the resin particles are in the form of aggregates.

In yet another aspect, the method comprises: (1) contacting ion-exchange resin particles having particle sizes larger than 50 μm with at least one therapeutic agent to form resin-therapeutic agent complexes; (2) micronizing the resin-therapeutic agent complexes by subjecting the resin-therapeutic agent complexes to a size reduction process one or more times to obtain micronized resin-therapeutic agent complexes having particle sizes equal to or less than 50 μm; and (3) admixing with a pharmaceutically acceptable carrier to yield a pharmaceutical composition having uniform dispersion of the resin-therapeutic agent complexes and less than 1 wt % of the resin particles are in the form of aggregates.

In some embodiments, the size reduction process is jet milling, media milling, microfluidizer milling, or high-pressure homogenization.

The therapeutic agent and the ion-exchange resin particles can be provided at a ratio of the therapeutic agent to the resin particle is in the range from 5:1 to 1:100 by weight, calculated on a moisture-free basis. In some embodiments, the ratio of the therapeutic agent to the resin particle is in the range from 2:1 to 1:20 by weight.

In some embodiments, the methods described above further comprise adding a dispersing agent before or after the step of micronizing, thereby when the pharmaceutical composition is re-dispersed in a liquid medium, uniform dispersion of the resin-therapeutic agent complexes is produced and less than 1 wt % of the micronized resin particles are in the form of aggregates.

In some embodiments, the dispersing agent is: (1) a water-soluble substance selected from the group consisting of water-soluble polymers, hydrophilic surfactants, sugars, such as gelatin, Arabic gum, agarose, polyvinyl pyrrolidone, hydroxypropyl methylcellulose, hydroxypropyl cellulose, sodium carboxymethyl cellulose, mannitol, lactose, sucrose, sodium lauryl sulfate, poloxamers, or combinations thereof, or (2) a solvent-soluble material selected from the group consisting of solvent-soluble polymers, lipophilic surfactants, phospholipids, fatty acids, such as polyvinyl pyrrolidone, phosphatidylcholine, phosphatidylethanolamine, stearate acid, oleic acid, or combinations thereof.

In some embodiments, the method further comprises formulating the pharmaceutical composition as a dosage form selected from the group consisting of suspension, dry powder for suspension, orally disintegrating tablets, mini-tablets/mini orally disintegrating tablets with the longest dimension typically less than or equal to 3 mm, chewable tablets, oral jelly, and oral gummies.

In some embodiments, the dosage form resulted from the above method of preparation is a liquid suspension comprising:
(a) between about 0.4% (w/w) and about 50% (w/w) (e.g., 0.4%, 0.6%, 0.8%, 1%, 4%, 8%, 12%, 16%, 20%, 24%, 28%, 32%, 36%, 40%, 44%, 48%, 50%) of the therapeutic agent;
(b) between about 0.4% (w/w) and about 84.6% (w/w) (e.g., 0.4%, 1%, 2%, 5%, 10%, 15%, 20%, 25%, 30%, 35%, 40%, 45%, 50%, 55%, 60%, 65%, 70%, 75%, 80%, 82%, 84%, 84.6%) of the micronized ion-exchange particles;
(c) between about 0% (w/w) and about 60% (w/w) of a dispersing aid or a dispersing agent;
(d) between about 0% (w/w) and about 40% (w/w) of a suspending agent or thickener;
(e) between about 0% (w/w) and about 20% (w/w) of an additional agent selected from the group consisting of a flavoring agent, a preservative, a pH adjusting agent, an antifoaming agent, a coloring agent, an antioxidant and a combination thereof; and
(f) between about 15% (w/w) and about 80% (w/w) of purified water.

In some embodiments, the dosage form resulted from the above method of preparation is a dry powder for suspension comprising:
(a) between about 0.4% (w/w) and about 50% (w/w) (e.g., 0.4%, 0.6%, 0.8%, 1%, 4%, 8%, 12%, 16%, 20%, 24%, 28%, 32%, 36%, 40%, 44%, 48%, 50%) of the therapeutic agent;
(b) between about 0.4% (w/w) and about 99.6% (w/w) (e.g., 0.4%, 1%, 2%, 5%, 10%, 15%, 20%, 25%, 30%, 35%, 40%, 45%, 50%, 55%, 60%, 65%, 70%, 75%, 80%, 82%, 84%, 86%, 88%, 90%, 94%, 96%, 98%, 99.6%) of the micronized ion-exchange particles;
(c) between about 0% (w/w) and about 60% (w/w) of a dispersing aid or a dispersing agent;
(d) between about 0% (w/w) and about 40% (w/w) of a thickener; and
(e) between about 0% (w/w) and about 20% (w/w) of an additional agent selected from the group consisting of a flavoring agent, a preservative, a pH adjusting agent, an antifoaming agent, a coloring agent, an antioxidant and a combination thereof.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows micronized Amberlite IRP 64 resin.

FIG. 2 shows micronized Amberlite IRP 69 resin.

FIG. 3 shows micronized Duolite AP143 resin.

FIG. 4 shows dry suspensions containing micronized resin-Guanfacine complexes with dispersing aids constituted in water.

FIG. 5 shows dry suspensions containing micronized resin-Guanfacine complexes with dispersing aids constituted in whole milk.

FIG. 6 shows dry suspensions containing micronized resin-Guanfacine complexes with dispersing aids constituted in soy milk.

FIG. 7 shows dry suspensions containing micronized resin-Guanfacine complexes with dispersing aids constituted in infant formula.

FIG. 8 shows dry suspensions containing micronized resin-Oseltamivir complexes with dispersing aids constituted in water.

FIG. 9 shows dry suspensions containing micronized resin-Oseltamivir complexes with dispersing aids constituted in whole milk.

FIG. 10 shows dry suspensions containing micronized resin-Oseltamivir complexes with dispersing aids constituted in soy milk.

FIG. 11 shows dry suspensions containing micronized resin-Oseltamivir complexes with dispersing aids constituted in infant formula.

FIG. 12 shows dry suspensions containing micronized resin-pramipexole complexes with dispersing aids constituted in water.

FIG. 13 shows dry suspensions containing micronized resin-pramipexole complexes with dispersing aids constituted in whole milk.

FIG. 14 shows dry suspensions containing micronized resin-pramipexole complexes with dispersing aids constituted in soy milk.

FIG. 15 shows dry suspensions containing micronized resin-pramipexole complexes with dispersing aids constituted in infant formula.

FIG. 16 shows dry suspensions containing micronized resin-ropinirole complexes with dispersing aids constituted in water.

FIG. 17 shows dry suspensions containing micronized resin-ropinirole complexes with dispersing aids constituted in whole milk.

FIG. 18 shows dry suspensions containing micronized resin-ropinirole complexes with dispersing aids constituted in soy milk.

FIG. 19 shows dry suspensions containing micronized resin-ropinirole complexes with dispersing aids constituted in infant formula.

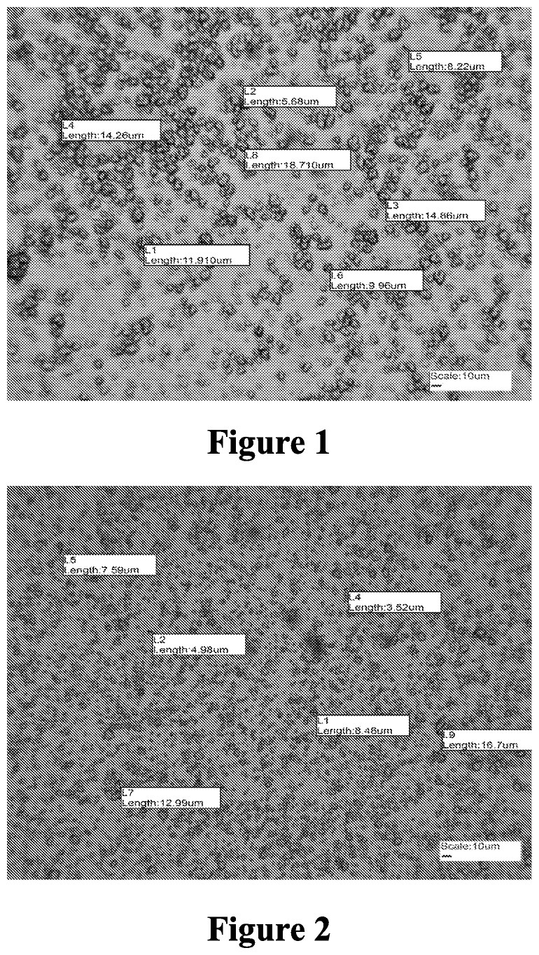

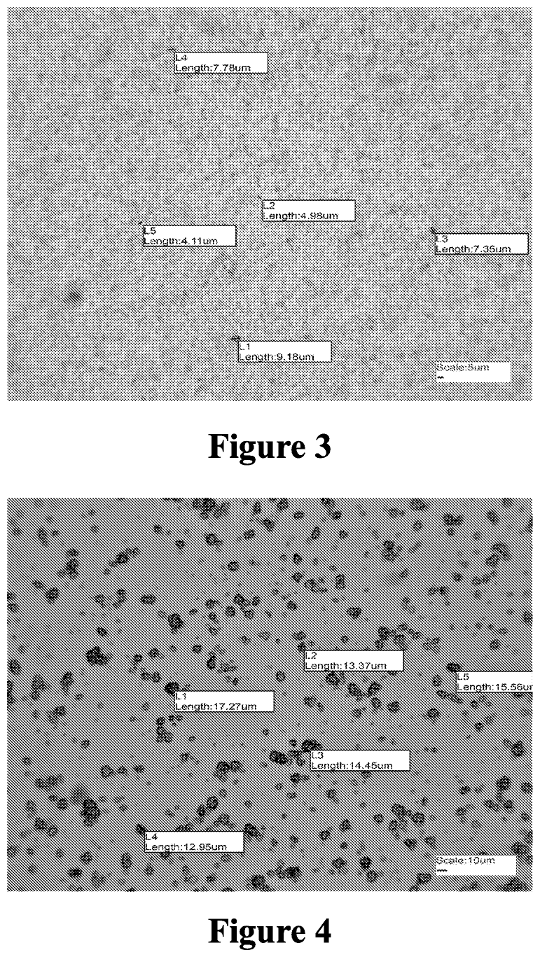

### DETAILED DESCRIPTION OF THE INVENTION

Commercially available ion-exchange resins which have a typical particle size of approximately 50-150 μm. Large particle sizes of commercial ion exchange resins produce large particle size resin-therapeutic agent complexes. When the resin-therapeutic agent complexes are further processed, the particle size of the final product is further increased. For example, U.S. Pat. No. 8,062,667 described polymer-coated resinate particles that can pass through 40 mesh (400 μm). U.S. Pat. No. 7,067,116 described using ion exchange resin to produce coated ion exchange resin-drug complex with average particle size ranging about 60 to about 250 μm. Particle size in such ranges causes gritty mouthfeel. Unpleasant mouthfeel caused by grittiness may affect children's adherence to medication and thus undermine the outcome of treatment, especially for younger end of the childhood age group spectrum, e.g., children less than two years old of age.

Large particle sizes of resin-therapeutic agent complexes can also have a negative effect on active uniformity of certain age-appropriate dosage forms, such as mini-tablet or orally disintegrating mini-tablet. These tablets are typically less than 3 mm in diameter and weigh about 5-30 mg per tablet. Mini-tablets and orally disintegrating mini-tablets are particularly suitable for pediatric patients and especially younger pediatric patients, such as infants, due to its dosing flexibility, fast disintegration, and ease of swallowing characteristics (Thomson S A, et al. Minitablets: new modality to deliver medicines to preschool-aged children. Pediatrics 2009; 123: e235-e8; Spomer N, et al. Acceptance of uncoated mini-tablets in young children: results from a prospective exploratory cross-over study. Arch Dis Child 2012; 97: 283-6). However, tablets in such size and weight pose huge challenge on content uniformity (i.e., individual content of the active ingredient in each tablet). Content uniformity is a critical quality attribute of these mini-tablets due to the requirement of dosing accuracy for pediatric patients such as infants. In the treatment when accurate dosing is critical, e.g., for Narrow Therapeutic Index (NTI) drugs, high uniformity is even more important. To achieve uniformity in mini-tablet, high blend uniformity and tight tablet weight control are a must. High blend uniformity can be achieved by uniformity dispersing drug substance in the blend by using conventional formulation means such as dispersing micronized drug substance or dissolving drug substance and spread them uniformly in granules/blend by various dry or wet granulation processes. However, such uniformity improvement means can exaggerate the unpleasant taste of the finely dispersed drug substance.

Large particle sizes of commercial ion exchange resins can also cause fast settlement in suspension formulations during storage, and it requires vigorous shaking to re-disperse the settled resins. The settlement of suspension results in potential drug uniformity issues. Since typical volume for a unit dose of suspension formulation is 5 to 10 ml, if a suspension is not uniform, accurate dosing cannot be assured. In the treatment when accurate dose adjustment is critical, e.g., for Narrow Therapeutic Index (NTI) drugs, an ununiform suspension brings in an additional challenge for medical professionals.

Further, when administrating medicine to children, dosage forms are frequently manipulated by crushing tablets or emptying capsule contents and then mix them with soft foods (e.g., yogurt, jam, etc.) or drink (e.g., juice) before giving to children to ingest. Such practice is problematic in many ways, such as dose loss or incomplete dose ingestion, unpleasant taste, unsuitable for modified release formulations, and unsuitable for children in younger age group such as neonates and infants less than two year's old. A better way of administering medicine to children is needed, such as a pharmaceutical dosage form that can be easily dispersible in milk, juice, and provides good mouthfeel, taste, and uniformity.

Current particle sizes of resinate formulations cannot serve the above purposes well. The grittiness and uniformity concern will prevent such formulation from being sprinkled, mixed, or suspended in soft foods or liquids, especially not suitable for sprinkled or suspended in milk for neonates or infants. The larger particle size of the current resinate formulations also cannot provide sufficient assurance of uniformity in tandem with taste concealing/masking feature in formulations such as mini-tablet/orally disintegrating mini-tablet, low unit dose volume suspension or powder for suspension during their manufacturing and use.

This disclosure provides a pharmaceutical composition, which comprises micronized ion-exchange resin particles and at least one therapeutic agent releasably bound to the micronized resin particles through ionic interaction to form resin-therapeutic agent complexes. The resin-therapeutic agent complexes have particle sizes less than 50 μm, and the pharmaceutical composition is formulated as a dosage form having uniform dispersion of the resin-therapeutic agent complexes and a reduced gritty mouthfeel for geriatric patients and pediatric patients, especially neonates and infants. It also provides more accurate dosing in the dose adjustment situation. In the case of mini-tablets, it provides benefits in both taste masking and improved content uniformity of individual mini-tablets. Thus, the disclosed micronized drug resinate represents an effective strategy for developing age-appropriate formulations, especially for pediatric and geriatric patients.

### I. Micronized Drug Resinate-Based Compositions

### A. Resin-Drug Complexes

In one aspect, this disclosure provides a pharmaceutical composition for oral administration. The pharmaceutical composition comprises micronized ion-exchange resin particles having particle sizes less than 50 μm, and at least one therapeutic agent releasably bound to the micronized resin particles through ionic interaction to form resin-therapeutic agent complexes (drug resinates). The resin-therapeutic agent complexes have particle sizes less than 50 μm, and the pharmaceutical composition is formulated as a dosage form (e.g., liquid dosage form or solid dosage form) that can produce uniform dispersion of the resin-therapeutic agent complexes with less than 10 wt % (e.g., less than 8 wt %, less than 6 wt %, less than 4 wt %, less than 2 wt %, less than 1 wt %) of the resin particles are in the form of aggregates. Due to the small particle sizes and uniform dispersion of the resin-therapeutic agent complexes, the taste of the therapeutic agent is substantially masked, and the dosage form has a reduced gritty mouth feel compared to a dosage form containing resin particles with particle sizes larger than 50 μm. In addition, due to the small particle sizes of the resin-therapeutic agent complexes, the drug content uniformity of certain dosage forms, i.e., mini-tablet/orally disintegrating mini-tablet, low unit dose volume suspension or powder for suspension, can be assured in tandem with taste concealing/masking feature.

The disclosed resin-therapeutic agent complexes, when formulated into oral suspension, due to its small particle size, have less tendency of forming sedimentation, easier re-dispersing, more uniformly distributed in the liquid media, can be more accurately dosed and can be dosed in small liquid volumes (e.g., <5 ml). The resin-therapeutic agent complexes-based oral suspension are particularly suitable for administering medicines that require flexible-dose adjustment to assure therapeutic benefit and minimize adverse effect, such as NPI drugs that require dose adjustment according to patient medical conditions (e.g., impaired liver or kidney functions), for individualized treatment, such as pediatric treatment in ICU, and for any situation when individualized dosing is required.

Further, the resin-therapeutic agent complexes can be administered to special groups of patients (e.g., pediatric or geriatric patients) by dispersing in soft foods, such as yogurt, jams, puddings, etc. or in liquids, such as juices, milk, water, honey, etc. It can also be added to infant formula for drug administration due to its taste-masking and good mouthfeel attributes.

In some embodiments, at least about 10%, 20%, 30%, 40%, 50% 60%, 70%, 80% or 90% of the resin-therapeutic agent complexes can be about 0.5 μm to about 50 μm in its largest dimension, e.g., about 1 μm to about 40 μm, about 1 μm to about 30 μm, about 1 μm to about 20 μm, or about 1 μm to about 10 μm in its largest dimension. In some embodiments, at least about 10%, 20%, 30%, 40%, 50% 60%, 70%, 80% or 90% of the resin-therapeutic agent complexes can be about 5 μm to about 50 μm in its largest dimension, e.g., about 5 μm to about 40 μm, about 5 μm to about 30 μm, about 5 μm to about 20 μm, or about 5 μm to about 10 μm in its largest dimension. In some embodiments, at least about 10%, 20%, 30%, 40%, 50% 60%, 70%, 80% or 90% of the resin-therapeutic agent complexes can be about 10 μm to about 50 μm in its largest dimension, e.g., about 10 μm to about 40 μm, about 10 μm to about 30 μm, or about 10 μm to about 20 μm.

In some embodiments, the resin-therapeutic agent complexes may have a largest dimension (e.g., diameter) of less than about 50 μm. In some embodiments, the resin-therapeutic agent complexes have a largest dimension of less than about 40 μm. In some embodiments, the resin-therapeutic agent complexes have a largest dimension of less than about 30 μm. In some embodiments, the resin-therapeutic agent complexes have a largest dimension of less than about 20 μm. In some embodiments, the resin-therapeutic agent complexes have a largest dimension of less than about 10 μm.

In some embodiments, the resin-therapeutic agent complexes can have an average diameter of between about 0.5 μm and about 50 μm. In some embodiments, the resin-therapeutic agent complexes can have an average diameter of between about 1 μm and about 40 μm. In some embodiments, the resin-therapeutic agent complexes can have an average diameter of between about 1 μm and about 30 μm. In some embodiments, the resin-therapeutic agent complexes can have an average diameter of between about 1 μm and about 20 μm. In some embodiments, the resin-therapeutic agent complexes can have an average diameter of between about 1 μm and about 10 μm. In some embodiments, the resin-therapeutic agent complexes can have an average diameter of between about 5 μm and about 50 μm. In some embodiments, the resin-therapeutic agent complexes can have an average diameter of between about 5 μm and about 40 μm. In some embodiments, the resin-therapeutic agent complexes can have an average diameter of between about 5 μm and about 30 μm. In some embodiments, the resin-therapeutic agent complexes can have an average diameter of between about 5 μm and about 20 μm. In some embodiments, the resin-therapeutic agent complexes can have an average diameter of between about 5 μm and about 10 μm. In some embodiments, the resin-therapeutic agent complexes can have an average diameter of between about 10 μm and about 50 μm. In some embodiments, the resin-therapeutic agent complexes can have an average diameter of between about 10 μm and about 40 μm. In some embodiments, the resin-therapeutic agent complexes can have an average diameter of between about 10 μm and about 30 μm. In some embodiments, the resin-therapeutic agent complexes can have an average diameter of between about 10 μm and about 20 μm.

The resin-therapeutic agent complexes can have a variety of different shapes including spheres, oblate spheroids, cylinders, ovals, ellipses, shells, cubes, cuboids, cones, pyramids, rods (e.g., cylinders or elongated structures having a square or rectangular cross-section), tetrapods (particles having four leg-like appendages), triangles, prisms, etc.

It may be desirable to use a population of the resin-therapeutic agent complexes that is relatively uniform in terms of size, shape, and/or composition so that each particle of the resin-therapeutic agent complexes has similar properties. For example, at least 80%, at least 90%, or at least 95% of the particles may have a diameter or largest dimension that falls within 5%, 10%, or 20% of the average diameter or largest dimension. In some embodiments, a population of the resin-therapeutic agent complexes may be heterogeneous with respect to size, shape, and/or composition.

In some embodiments, one or more substantially uniform populations of the resin-therapeutic agent complexes are used, e.g., 2, 3, 4, 5, or more substantially uniform populations having distinguishable properties (e.g., size, optical property) or associated with different therapeutic agents. For example, each population of particles may be associated with one therapeutic agent. In some embodiments, the disclosed pharmaceutical composition may include two or more populations of the resin-therapeutic agent complexes, each of which is associated with one therapeutic agent. It will be appreciated that a combination of two or more populations having distinguishable properties can be considered to be a single population.

In some embodiments, the pharmaceutical composition further comprises a dispersing agent, thereby when the pharmaceutical composition is re-dispersed in a liquid medium, uniform dispersion of the resin-therapeutic agent complexes is produced and less than 20 wt % (e.g., less than 15 wt %, less than 10 wt %, less than 8 wt %, less than 6 wt %, less than 4 wt %, less than 2 wt %, less than 1 wt %) of the resin particles are in the form of aggregates.

The dispersing agent can be (1) a water-soluble substance selected from the group consisting of water-soluble polymers, hydrophilic surfactants, sugars, such as gelatin, Arabic gum, agarose, sodium alginate, polyvinyl pyrrolidone, hydroxypropyl methylcellulose, hydroxypropyl cellulose, sodium carboxymethyl cellulose, mannitol, lactose, sucrose, sodium lauryl sulfate, poloxamers, or combinations thereof, or (2) a water-insoluble but hydrophilic and swellable material commonly referred to as “disintegratant” selected from the group consisting of cross-linked or non-crosslinked synthetic or natural polymers, such as sodium starch glycolate, cross-linked polyvinylpyrrolidone, croscarmellose sodium, and alginic acid. For a pharmaceutical composition intended to be dispersed into an organic medium, the dispersing agent can be a solvent-soluble material selected from the group consisting of solvent-soluble polymers, lipophilic surfactants, phospholipids, fatty acids, such as polyvinyl pyrrolidone, phosphatidylcholine, phosphatidylethanolamine, stearate acid, oleic acid, or combinations thereof.

(1) Ion-Exchange Resins

Ion-exchange resins suitable for use in these preparations are water-insoluble and comprise a preferably pharmacologically inert organic and/or inorganic matrix containing functional groups that are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix may include silica gel modified by the addition of ionic groups. Covalently bound ionic groups may be strongly acidic (e.g., sulfonic acid, phosphoric acid), weakly acidic (e.g., carboxylic acid), strongly basic (e.g., primary amine), weakly basic (e.g., quaternary ammonium), or a combination of acidic and basic groups. In general, the types of ion exchangers suitable for use in ion-exchange chromatography and for such applications as deionization of water are suitable for use in the present disclosure. Such ion-exchangers are described by H. F. Walton in “Principles of Ion Exchange” (pp: 312-343) and “Techniques and Applications of Ion-Exchange Chromatography” (pp: 344-361) in Chromatography. (E. Hoffmann), van Nostrand Reinhold Company, New York (1975). Ion exchange resins that can be used in the present invention have exchange capacities of about 6 milliequivalents (meq)/gram or below (e.g., about 5.5 meq/gram, about 5 meq/gram, about 4.5 meq/gram, about 4 meq/gram, about 3.5 meq/gram, about 3 meq/gram).

Commercially available ion-exchange resins having a regular or irregular shape and diameters up to about 1,000 microns are gritty in liquid dosage forms. Thus, according to one aspect of this disclosure, ion-exchange resin particles may be subject to a size reduction process (e.g., milling, homogenization) to obtain micronized resin particles with particle sizes less than or equal to 50 μm.

In some embodiments, at least about 10%, 20%, 30%, 40%, 50% 60%, 70%, 80% or 90% of the resin particles can be about 0.5 μm to about 50 μm in its largest dimension, e.g., about 1 μm to about 40 μm, about 1 μm to about 30 μm, about 1 μm to about 20 μm, or about 1 μm to about 10 μm in its largest dimension. In some embodiments, at least about 10%, 20%, 30%, 40%, 50% 60%, 70%, 80% or 90% of the resin particles can be about 5 μm to about 50 μm in its largest dimension, e.g., about 5 μm to about 40 μm, about 5 μm to about 30 μm, about 5 μm to about 20 μm, or about 5 μm to about 10 μm in its largest dimension. In some embodiments, at least about 10%, 20%, 30%, 40%, 50% 60%, 70%, 80% or 90% of the resin particles can be about 10 μm to about 50 μm in its largest dimension, e.g., about 10 μm to about 40 μm, about 10 μm to about 30 μm, or about 10 μm to about 20 μm.

In some embodiments, the resin particles may have a largest dimension (e.g., diameter) of less than about 50 μm. In some embodiments, the resin particles have a largest pediatric of less than about 40 μm. In some embodiments, the resin particles have a largest dimension of less than about 30 μm. In some embodiments, the resin particles have a largest dimension of less than about 20 μm. In some embodiments, the resin particles have a largest dimension of less than about 10 μm.

In some embodiments, the resin particles can have an average diameter of between about 0.5 μm and about 50 μm. In some embodiments, the resin particles can have an average diameter of between about 1 μm and about 40 μm. In some embodiments, the resin particles can have an average diameter of between about 1 μm and about 30 μm. In some embodiments, the resin particles can have an average diameter of between about 1 μm and about 20 μm. In some embodiments, the resin particles can have an average diameter of between about 1 μm and about 10 μm. In some embodiments, the resin particles can have an average diameter of between about 5 μm and about 50 μm. In some embodiments, the resin particles can have an average diameter of between about 5 μm and about 40 μm. In some embodiments, the resin particles can have an average diameter of between about 5 μm and about 30 μm. In some embodiments, the resin particles can have an average diameter of between about 5 μm and about 20 μm. In some embodiments, the resin particles can have an average diameter of between about 5 μm and about 10 μm. In some embodiments, the resin particles can have an average diameter of between about 10 μm and about 50 μm. In some embodiments, the resin particles can have an average diameter of between about 10 μm and about 40 μm. In some embodiments, the resin particles can have an average diameter of between about 10 μm and about 30 μm. In some embodiments, the resin particles can have an average diameter of between about 10 μm and about 20 μm.

It may be desirable to use a population of the resin particles that is relatively uniform in terms of size, shape, and/or composition so that each particle of the resin particles has similar properties. For example, at least 80%, at least 90%, or at least 95% of the particles may have a diameter or largest dimension that falls within 5%, 10%, or 20% of the average diameter or largest dimension.

In some embodiments, a population of the resin particles may be heterogeneous with respect to size, shape, and/or composition. Both regularly and irregularly shaped resin particles may be used for the present invention. Regularly shaped particles are those particles that substantially conform to geometric shapes such as spherical, elliptical, cylindrical and the like, which are exemplified by Dow XYS-40010.00 and Dow XYS-40013.00 (The Dow Chemical Company). Irregularly shaped particles are all particles not considered to be regularly shaped, such as particles with amorphous shapes and particles with increased surface areas due to surface channels or distortions. Irregularly shaped ion-exchange resins of this type are exemplified by Amberlite IRP-69, Amberlite IRP-64, DUOLITE AP143/1083 (Rohm and Haas).

Suitable ion-exchange resins include anion exchange resins, such as have been described in the art and are commercially available. These resins are particularly well suited for use with acidic drugs including, e.g., nicotinic acid, mefenamic acid, indomethacin, diclofenac, repaglinide, ketoprofen, ibuprofen, valproic acid, lansoprazole, ambroxol, omeprazole, acetaminophen, topiramate, and carbamazepine, pentobarbital, warfarin, triamterene, and prednisolone, as well as prodrugs, salts, isomers, polymorphs, and solvates thereof, as well as other drugs identified herein and/or known in the art.

An example of an anion exchange resin is a cholestyramine resin, a strong base type 1 anion exchange resin powder with a polystyrene matrix and quaternary ammonium functional groups. The exchangeable anion is generally chloride which can be exchanged for, or replaced by, virtually any anionic species. A commercially available Cholestyramine resin is PUROLITE™ A430MR resin. As described by its manufacturer, this resin has an average particle size range of less than 150 μm, a pH in the range of 4-6, and an exchange capacity of 1.8-2.2 eq/dry gm. Another pharmaceutical-grade cholestyramine resin is available as DUOLITE™ AP143/1094, described by the manufacturer as having a particle size in the range of 95%, less than 100 microns and 40%, less than 50 microns. The commercial literature from the suppliers of these and other resin is incorporated herein by reference (PUROLITE A-430 MR; DOW Cholestryramine USP, Form No. 177-01877-204, Dow Chemical Company; DUOLITE AP143/1083, Rohm and Haas Company, IE-566EDS). Another example of basic resins useful in this invention includes anion exchange resin such as Duolite AP143/1093 (colestyramine resin). Duolite AP143/1093 has a sodium glycocholate exchange capacity of 1.8-2.2 meq/g and a particle size range of no less than 95% less than 100 microns and no less than 40% less than 50 microns.

Cation exchange resins (e.g., AMBERLITE IRP-69) are well suited for use with drugs and other molecules having a cationic functionality, including, e.g., acycloguanosine, tinidazole, deferiprone, cimetidine, oxycodone, remacemide, nicotine, morphine, guanfacine, hydrocodone, rivastigmine, dextromethorphan, propanolol, betaxolol, 4-aminopyridine, chlorpheniramine, paroxetine, duloxetine HCl, atomoxetine HCl, risperidone, atovaquone, oseltamivir, esmolol, naloxone, phenylpropanolamine, gemifloxacin, oxymorphone, hydromorphone, nalbuphine, and O-desmethylvenlafaxine, as well as prodrugs, salts, isomers, polymorphs, and solvates thereof, as well as other drugs identified herein and/or known in the art. Cationic exchange resins are readily selected for the use of these basic drugs or other drugs identified herein and/or are those which are known to those of skill in the art.

Representative acidic resins useful in this invention include pharmaceutical-grade strongly acidic cation exchange resin such as AMBERLITE IRP-69 (sodium polystyrene sulfonate), and weakly acidic cation exchange resin Amberlite IRP-64 (polymethacrylic acid), obtained from Rohm and Haas/Dow. Amberlite IRP-69 has an ion exchange capacity of about 110 to 135 mg/g potassium and a particle size range of 10.0-25.0% larger than 75 microns and no more than 1.0% larger than 150 microns. Amberlite IRP-64 has an ion exchange capacity of about 10.0 meq/g and a particle size range of <=1.0% larger than 150 microns, 15.0-30.0% larger than 75 microns, and <=70.0% smaller than 50 microns. Its exchange capacity is normally within the range of approximately 3 to 4 meq/g of dry resin.

The selected ion-exchange resins may be further treated by the manufacturer or the purchaser to maximize the safety for pharmaceutical use or for improved performance of the compositions. Impurities present in the resins may be removed or neutralized by the use of common chelating agents, anti-oxidants, preservatives such as disodium edetate, sodium bisulfite, and so on by incorporating them at any stage of preparation either before complexation or during complexation or thereafter. These impurities along with their chelating agent to which they have bound may be removed before further treatment of the ion exchange resin with a release retardant and diffusion barrier coating.

In some embodiments, the resin particle is an anionic ion-exchange resin particle or a cationic ion-exchange resin particle. The resin particle can be a cross-linked sulfonated polystyrene ion-exchange resin (e.g., Amberlite IRP-69), a cross-linked methacrylic acid and divinylbenzene copolymer ion-exchange resin (e.g., Amberlite IRP-64), a cross-linked copolymer of diethylenetriamine and 1-chloro-2,3-epoxy propane ion-exchange resin (e.g., Colestipol hydrochloride), or a cross-linked copolymer of styrene and divinylbenzene with quaternary ammonium functionality ion-exchange resin (e.g., Duolite AP143/1093). In some embodiments, the resin particle is Amberlite IRP-69, Amberlite IRP-64, Colestipol hydrochloride, or Duolite AP143/1093.

In some embodiments, the resin particle has an ion-exchange capacity of less than 6 (e.g., less than 5, less than 4, less than 3, less than 2) milliequivalents per gram (meq/g) of dry resin.

(2) Therapeutic Agents

The amount of the therapeutic agent (or drug) loaded onto the ion exchange resin may be in the range of from about 0.00001% to about 90% by weight of the resin-therapeutic agent complex. In some embodiments, the amount of the therapeutic agent loaded onto the ion exchange resin is at least 0.00001% and in the range from about 0.00001% to about 80% by weight of the resin-therapeutic agent complex. In some embodiments, the amount of the therapeutic agent loaded onto the ion exchange resin is about 0.00001% to about 70% by weight of the resin-therapeutic agent complex. In some embodiments, the amount of the therapeutic agent loaded onto the ion exchange resin is about 0.00001% to about 60% by weight of the resin-therapeutic agent complex. In some embodiments, the amount of the therapeutic agent loaded onto the ion exchange resin is about 0.00001% to about 50% by weight of the resin-therapeutic agent complex. In some embodiments, the amount of the therapeutic agent loaded onto the ion exchange resin is about 0.00001% to about 40% by weight of the resin-therapeutic agent complex.

In some embodiments, the ratio of the therapeutic agent to the resin particle is in the range from 0.0000001:1 to 3:1 by weight, calculated on a moisture-free, free acid or base basis. In some embodiments, the ratio of the therapeutic agent to the resin particle is in the range from 0.000001:1 to 1:1 by weight. In some embodiments, the ratio of the therapeutic agent to the resin particle is in the range from 0.00001:1 to 1:1 by weight. In some embodiments, the ratio of the therapeutic agent to the resin particle is in the range from 0.0001:1 to 1:1 by weight. In some embodiments, the ratio of the therapeutic agent to the resin particle is in the range from 0.001:1 to 1:1 by weight.

The therapeutic agents that are suitable for use in these preparations in terms of chemical nature are acidic, basic, amphoteric, or zwitterionic molecules. Such therapeutic agents include small molecules, and selected larger molecules as well, including chemical moieties and biologicals, such as, e.g., a protein or a fragment thereof (e.g., a peptide, polypeptide, etc.), enzyme, antibody or antibody fragment.

The therapeutic agent is selected based on inclusion in the molecule of a group, such as an amino group, which will readily bind to a charged complexing agent such as an ion-exchange resin. Any therapeutic agent that bears an acidic or a basic functional group, for example, an amine, imine, imidazolyl, guanidine, piperidinyl, pyridinyl, quaternary ammonium, or other basic group, or a carboxylic, phosphoric, phenolic, sulfuric, sulfonic or other acidic group, can be bound to a resin of the opposite charge. Representative therapeutic agent agents are described in, for example, WO 98/18610 by Van Lengerich; U.S. Pat. No. 6,512,950 to Li et al. and U.S. Pat. No. 4,996,047 to Kelleher et al.

Examples of therapeutic agents that bear acidic or basic functional groups and thus may be complexed with a binding resin include, but are not limited to Acetylsalicylic acid, Alendronic acid, Alosetron, Amantadine, Amlopidine, Anagrelide, Argatroban, Atomoxetine, Atrovastatin, Azithromycin dehydrate, Balsalazide, Bromocriptan, Bupropion, Candesartan, Carboplatin, Ceftriaxone, Clavulonic acid, Clindamycin, Cimetadine, Dehydrocholic (acid), Dexmethylphenidate, Diclofenac, Dicyclomine, Diflunisal, Diltiazem, Donepezil, Doxorubicin, Doxepin, Epirubicin, Etodolic acid, Ethacrynic acid, Fenoprofen, Fluoxetine, Furosemide, Guanfacine, Gemfibrozil, Hydroxyzine, Ibuprofen, Imipramine, Levothyroxine, Liothyronine, Maprolitline, Meclizine, Methadone, Methylphenidate, Minocycline, Mitoxantone, Moxifloxacin, Mycophenolic acid, Naproxen, Niflumic acid, Ofloxacin, Ondansetron, Oseltamivir, Pantoprazole, Paroxetine, Pergolide, Pramipexole, Phenytoin, Pravastain, Probenecid, Rabeprazole, Risedronic acid, Retinoic acid, Ropinirole, Selegiline, Sulindac, Tamsulo sin, Telmisertan, Terbinafine, Theophyline, Tiludronic Acid, Tinzaparin, Ticarcillin, Valproic acid, Salicylic acid, Sevelamer, Ziprasidone, Zoledronic acid, Acetophenazine, Albuterol, Almotriptan, Amitriptyline, Amphetamine, Atracurium, Beclomethasone, Benztropine, Biperiden, Bo sentan, Bromodiphenhydramine, Brompheniramine carbinoxamine, Caffeine, Capecitabine, Carbergoline, Cetirizine, Chlocylizine, Chlorpheniramine, Chlorphenoxamine, Chlorpromazine, Citalopram, Clavunate potassium, Ciprofloxacin, Clemastine, Clomiphene, Clonidine, Clopidogrel, Codeine, Cyclizine, Cyclobenzaprine, Cyproheptadine, Delavirdine, Diethylpropion, Divalproex, Desipramine, Dexmethylphenidate, Dexbrompheniramine, Dexchlopheniramine, Dexchlor, Dextroamphetamine, Dexedrine, Dextromethorphan, Diphemanil methylsulphate, Diphenhydramine, Dolasetron, Doxylamine, Enoxaparin, Ergotamine, Ertepenem, Eprosartan, Escitalopram, Esomeprazole, Fenoldopam, Fentanyl, Fexofenadine, Fluvastatin, Fluphenazine, Fluticasone, Fosinopril, Frovatriptan, Gabapentin, Galatamine, Gatifloxacin, Gemcitabine, Haloperidol, Hyalurondate, Hydrocodone, Hydroxychloroquine, Hyoscyamine, Imatinib, Imipenem, Ipatropin, Lisinopril, Leuprolide, Levopropoxyphene, Losartan, Mesalamine, Mepenzolate, Meperidine, Mephentermine, Mesalimine, Mesoridazine, Metaproteranol, Metformin, Methdialazine, Methscopolamine, Methysergide, Metoprolol, Metronidazole, Mibefradil, Montelukast, Morphine, Mometasone, Naratriptan, Nelfinavir, Nortriptylene, Noscapine, Nylindrin, Orphenadrine, Oseltamivir, Oxybutynin, Papaverine, Pentazocine, Phendimetrazine, Phentermine, Pioglitazone, Pilocarpine, Prochloroperazine, Pyrilamine, Quetapine, Ranitidine, Rivastigmine, Rosiglitazone, Salmetrol, Sertaline, Sotalol, Sumatriptan, Tazobactam, Tacrolimus, Tamoxifen, Ticlopidine, Topiramate, Tolterodine, Triptorelin, Triplennamine, Triprolidine, Tramadol, Trovofloxacin, Ursodiol, Promazine, Propoxyphene, Propanolol, Pseudoephedrine, Pyrilamine, Quinidine, Oxybate sodium, Sermorelin, Tacrolimus, Tegaseroid, Teriparatide, Tolterodine, Triptorelin pamoate, Scoplolamine, Venlafaxine, Zamivir, Aminocaproic acid, Amino salicylic acid, Hydromorphone, Isosuprine, Levorphanol, Melhalan, Nalidixic acid, and Para-amino salicylic acid.

In some embodiments, the therapeutic agent is acidic, including the therapeutic agent that contains a carboxyl group. In some embodiments, the therapeutic agent can be one of dehydrocholic acid, diflunisal, ethacrynic acid, fenoprofen, furosemide, gemfibrozil, ibuprofen, naproxen, phenytoin, probenecid, sulindac, theophylline, salicylic acid, and acetylsalicylic acid.

In some embodiments, wherein the therapeutic agent is basic, including the therapeutic agent contains an amine group. In some embodiments, the therapeutic agent can be one of acetophenazine, amitriptyline, amphetamine, benztropine, biperiden, bromodiphenhydramine, brompheniramine, carbinoxamine, chlorcyclizine, chlorpheniramine, chlorphenoxamine, chlorpromazine, clemastine, clomiphene, clonidine, codeine, cyclizine, cyclobenzaprine, cyproheptadine, desipramine, dexbrompheniramine, dexchlorpheniramine, dextroamphetamine, dextromethorphan, dicyclomine, diphemanil, diphenhydramine, doxepin, doxylamine, ergotamine, fluphenazine, haloperidol, hydrocodone, hydroxychloroquine, hydroxyzine, hyoscyamine, imipramine, levopropoxyphene, maprotiline, meclizine, mepenzolate, meperidine, mephentermine, mesoridazine, methadone, methdilazine, methscopolamine, methysergide, metoprolol, nortriptyline, no scapine, nylindrin, orphenadrine, papaverine, pentazocine, phendimetrazine, phentermine, phenylpropanolamine, pyrilamine, tripelennamine, triprolidine, promazine, propoxyphene, propanolol, pseudoephedrine, pyrilamine, quinidine, scopolamine, dextromethorphan, chlorpheniramine, and codeine.

In some embodiments, the therapeutic agent is amphoteric. In some embodiments, the therapeutic agent can be one of aminocaproic acid, amino salicylic acid, hydromorphone, isoxsuprine, levorphanol, melphalan, morphine, nalidixic acid, and paraamino salicylic acid.

In some embodiments, the therapeutic agent is selected from the group consisting of analeptic agents; analgesic agents; anesthetic agents; antiasthmatic agents; antiarthritic agents; anticancer agents; anticholinergic agents; anticonvulsant agents; antidepressant agents, antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism active agents; antipruritic agents; antipsychotic agents; antipyretic agents; antispasmodic agents; antitubercular agents; antiulcer agents; antiviral agents; anxiolytic agents; appetite Suppressants (anorexic agents); attention deficit disorder and attention deficit hyper activity disorder active agents; cardiovascular agents including calcium channel blockers and antianginal agents; central nervous system (CNS) agents; beta-blockers and antiar rhythmic agents; central nervous system stimulants; diuretics; genetic materials; hormonolytics; hypnotics; hypoglycemic agents; immuno suppressive agents; muscle relaxants; narcotic antagonists; nicotine; nutritional agents; parasym patholytics; peptide active agents; psychoStimulants; sedatives; Sialagogues, steroids; Smoking cessation agents; Sympathomimetics; tranquilizers; vasodilators; beta-agonist; tocolytic agents; and combinations thereof.

Further, pharmaceutically active metabolites or pharmaceutically acceptable prodrugs, salts, isomers, polymorphs, and solvates of the therapeutic agents identified above are also useful in the present invention. In addition, the free base of the salts specifically listed may be substituted with other pharmaceutically acceptable salts, or use as the free base, or a prodrug form.

### B. Dosage Forms

The disclosed pharmaceutical compositions can be formulated into various dosage forms, such as suspension (e.g., liquid suspension), dry powder for suspension, orally disintegrating tablets, mini-tablets or orally disintegrating mini-tablets with the longest dimension less than or equal to 3 mm, chewable tablets, oral jelly, and oral gummies.

In yet another embodiment, the formulations may contain more than one therapeutic agent. For example, the formulation may contain a first resin-therapeutic agent complex in combination with another therapeutic agent which may be in the same or a second resin-therapeutic agent complex. In still another example, the formulation may contain a resin-therapeutic agent complex in combination with one or more therapeutic agent which are not in a resin-therapeutic agent complex.

The resin-therapeutic agent complex may be formulated for delivery by any suitable route including, e.g., orally, topically, transdermally, sublingually, rectally, transbuccally, or vaginally. Preferably, the complex is formulated for oral delivery.

The pharmaceutical composition containing the disclosed micronized resin-therapeutic agent complex may be stored for future use or promptly formulated with conventional pharmaceutically acceptable carriers to prepare finished ingestible compositions for delivery orally, nasogastric tube, or via other means. The compositions according to this invention may, for example, take the form of liquid preparations such as suspensions, dry powder for suspension, or other solid preparations such as capsules, tablets, caplets, sublinguals, powders, wafers, strips, gels, including liquid gels, etc. In one embodiment, a tablet is formulated as an orally disintegrating tablet or orally disintegrating mini-tablet. Such orally dissolving tablets may disintegrate in the mouth in less than about 60 seconds.

The pharmaceutical composition may be formulated using conventional pharmaceutically acceptable carriers or excipients and well-established techniques. Without being limited thereto, such conventional carriers or excipients include diluents, binders and adhesives (i.e., cellulose derivatives and acrylic derivatives), lubricants (i.e., magnesium or calcium stearate, or vegetable oils, polyethylene glycols, talc, sodium lauryl sulfate, polyoxyethylene monostearate), thickeners, solubilizers, humectants, disintegrants, colorants, flavorings, stabilizing agents, sweeteners, and miscellaneous materials such as buffers and adsorbents in order to prepare a particular pharmaceutical composition. The stabilizing agents may include preservatives and anti-oxidants, amongst other components which will be readily apparent to one of ordinary skill in the art.

Suitable thickeners include, e.g., tragacanth; xanthan gum; bentonite; starch; acacia and lower alkyl ethers of cellulose (including the hydroxy and carboxy derivatives of the cellulose ethers). Examples of cellulose include, e.g., hydroxypropyl cellulose, hydroxypropyl methylcellulose, sodium carboxymethylcellulose, microcrystalline cellulose (MCC), and MCC with sodium carboxyl methylcellulose. In one embodiment, tragacanth is used and incorporated in an amount of from about 0.1 to about 1.0% weight per volume (w/v) of the composition, and more preferably about 0.5% w/v of the composition. Xanthan gum is used in the amount of from about 0.025 to about 0.5% w/v and preferably about 0.25% w/v.

Flocculating agents such as aluminum sulfate, carbopols, potassium acid phosphate, veegum, bentonite, etc. Wetting agents such as sodium lauryl sulfate, benzalkonium chloride, Spans, Tweens, etc., as described in standard texts also may be employed in the inventive compositions to facilitate the dispersion of any hydrophobic ingredients. The concentration of wetting agents in the composition should be selected to achieve optimum dispersion of the ingredient within the composition with the lowest feasible concentration of the wetting agent. It should be appreciated that an excess concentration of wetting agent may cause the composition, as a syrup suspension, to flocculate. Those skilled in the art are well versed in suitable empirical methods to determine the appropriate wetting agents and concentrations to achieve optimum dispersion and avoid caking. Those skilled in the art are well versed in suitable empirical methods to determine the appropriate wetting agents and concentrations to achieve optimum dispersion and avoid flocculation. Suitable wetting agents are listed in the US Pharmacopeia 29.

The drug resinates compositions also include a humectant to give the liquid greater viscosity and stability. Suitable humectants useful in the formulations of the present invention include glycerin, polyethylene glycol, propylene glycol and mixtures thereof, preferably polyethylene glycol is used and incorporated in an amount of from about 5% to about 20% w/v of the composition and preferably in an amount of from about 5% to about 15% w/v of the composition and most preferably in an amount of about 8% w/v of the composition.

The oral liquid compositions may also comprise one or more surfactants in amounts of up to about 5.0% w/v and preferably from about 0.02% to about 3.0% w/v of the total formulation. The surfactants useful in the preparation of the finished pharmaceutical compositions of the present invention are generally organic materials which aid in the stabilization and dispersion of the ingredients in aqueous systems for a suitable homogenous composition. Preferably, the surfactants of choice are non-ionic surfactants such as poly(oxyethylene) sorbitan monooleate and sorbitan monooleate. These are commercially known as TWEENS and SPANS and are produced in a wide variety of structures and molecular weights. Whereas any one of a number of surfactants may be used, preferably a compound from the group comprising polysorbate copolymers (sorbitan-mono-9-octadecenoate-poly(oxy-1,2-ethanediol)) is employed. This compound is also added functions to keep any flavors and sweeteners homogeneously dissolved and dispersed in solution. Suitable polysorbates include polysorbate 20, polysorbate 40, polysorbate 80, and mixtures thereof. Most preferably, polysorbate 80 is employed. The surfactant component will comprise from about 0.01% to about 2.0% w/v of the total composition and preferably will comprise about 0.1% w/v of the total weight of the composition.

A second emulsifier/surfactant useful in combination with polysorbates may be employed and is preferably a poloxamer such as Poloxamer 407. Poloxamer 407 has an HLB (hydrophilic/lipophilic balance) of about 22 and is sold under the tradename Pluronic-F127 (BASF-NJ). The two surfactants can be employed in substantially equivalent amounts. For example, the Poloxamer 407 and polysorbate 80 may each be employed together at levels of approximately from about 0.02% to about 4.0% w/v of the total weight of the formulation.

Aqueous suspensions may be obtained by dispersing the drug-ion exchange resin compositions in a suitable aqueous vehicle, optionally with the addition of suitable viscosity-enhancing agent(s) (e.g., cellulose derivatives, xanthan gum, etc.). Non-aqueous suspensions may be obtained by dispersing the foregoing compositions in a suitable non-aqueous based vehicle, optionally with the addition of suitable viscosity-enhancing agent(s) (e.g., hydrogenated edible fats, aluminum stearate, etc.). Suitable non-aqueous vehicles include, for example, almond oil, Arachis oil, soybean oil or fractionated vegetable oils such as fractionated coconut oil.

The pharmaceutical composition may also be formulated with a preservative. Useful preservatives include, but are not limited to, sodium benzoate, benzoic acid, potassium sorbate, salts of edetate (also known as salts of ethylenediaminetetraacetic acid, or EDTA, such as disodium EDTA), parabens (e.g., methyl, ethyl, propyl or butyl-hydroxybenzoate, etc.), and sorbic acid. Amongst useful preservatives include chelating agents, some of which are listed above and other chelating agents, e.g., nitrilotriacetic acid (NTA); ethylenediaminetetracetic acid (EDTA), hydroxyethylethylenediaminetriacetic acid (HEDTA), diethylenetriaminepentaacetic acid (DPTA), 1,2-Diaminopropanetetraacetic acid (1,2-PDTA); 1,3-Diaminopropanetetraacetic acid (1,3-PDTA); 2,2-ethylenedioxybis[ethyliminodi(acetic acid)] (EGTA); 1,10-bis(2-pyridylmethyl)-1,4,7,10-tetraazadecane (BPTETA); ethylenediamine (EDAMINE); Trans-1,2-diaminocyclohexane-N,N,N′,N′-tetraacetic acid (CDTA); ethylenediamine-N,N′-diacetate (EDDA); phenazine methosulphate (PMS); 2,6-Dichloro-indophenol (DCPIP); Bis(carboxymethyl)diaza-18-crown-6 (CROWN); porphine; chlorophyll; dimercaprol (2,3-Dimercapto-1-propanol); citric acid; tartaric acid; fumaric acid; malic acid; and salts thereof. The preservatives listed above are exemplary, but each preservative must be evaluated in each formulation, to assure the compatibility and efficacy of the preservative. Methods for evaluating the efficacy of preservatives in pharmaceutical formulations are known to those skilled in the art. Additional examples of preservatives are the paraben preservatives including, methyl, ethyl, propyl, and butylparaben. Methyl and propylparaben are most preferable. Preferably, both methyl and propylparaben are present in the formulation in a ratio of methylparaben to propylparaben of from about 2.5:1 to about 16:1, preferably 9:1.

In the instance where auxiliary sweeteners are utilized, the formulation may include those sweeteners well known in the art, including both natural and artificial sweeteners. Thus, additional sweeteners may be chosen from the following non-limiting list: water-soluble sweetening agents such as monosaccharides, disaccharides, and polysaccharides such as xylose, ribose, glucose, mannose, galactose, fructose, high fructose corn syrup, dextrose, sucrose, sugar, maltose, partially hydrolyzed starch, or corn syrup solids and sugar alcohols such as sorbitol, xylitol, mannitol, and mixtures thereof; In general, the amount of sweetener will vary with the desired amount of sweeteners selected for a particular liquid formulation. This amount will normally be 0.001% to about 90% by weight, per volume of the final liquid composition, when using an easily extractable sweetener. The water-soluble sweeteners described above, are preferably used in amounts of about 5% to about 70% by weight per volume, and most preferably from about 10% to about 50% by weight per volume of the final liquid composition. In contrast, the artificial sweeteners [e.g., sucralose, acesulfame K, and dipeptide based sweeteners] are used in amounts of about 0.005% to about 5.0% and most preferably about 0.01% to about 2.5% by weight per volume of the final liquid composition. These amounts are ordinarily necessary to achieve a desired level of sweetness independent from the flavor level achieved from flavor oils.

Suitable flavorings include both natural and artificial flavors, and mints such as peppermint, menthol, artificial vanilla, cinnamon, various fruit flavors, both individual and mixed, essential oils (i.e., thymol, eucalyptol, menthol, and methyl salicylate) and the like are contemplated. The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. The flavorings are generally utilized in amounts that will vary depending upon the individual flavor, and may, for example, range in amounts of about 0.01 to about 3% by weight per volume of the final composition weight.

The colorants useful in the present invention, include the pigments such as titanium dioxide that may be incorporated in amounts of up to about 1% by weight per volume, and preferably up to about 0.6% by weight per volume. Also, the colorants may include dyes suitable for food, drug, and cosmetic applications, and known as D&C and F.D. & C. dyes and the like. The materials acceptable for the foregoing spectrum of use are preferably water-soluble. Illustrative examples include indigoid dye, known as F.D. & C. Blue No. 2, which is the disodium salt of 5,5′indigotindisulfonic acid. Similarly, the dye known as F.D. & C. Green No. 1 comprises a triphenylmethane dye and is the monosodium salt of 4-[4-N-ethyl p-sulfobenzylamino)diphenylmethylene]-[1-(N-ethyl-N-p-sulfoniumbenzyl)-2,5-cyclohexadienimine]. A full recitation of all F.D. & C. and D. & C. and their corresponding chemical structures may be found in the Kirk-Othmer Encyclopedia of Chemical Technology, in Volume 5, at Pages 857-884, which text is accordingly incorporated herein by reference.

Suitable oils and fats that are usable would include partially hydrogenated vegetable or animal fats, such as coconut oil, palm kernel oil, beef tallow, lard, and the like. These ingredients are generally utilized in amounts up to about 7.0% by weight, and preferably up to about 3.5% by weight of the final product.

Antioxidants such as ascorbic acid, sodium sulfite, sodium bisulfate, nordihydroguaretic acid (NDGA), ethyl, propyl and octyl gallates, etc. in suitable proportion as individuals or in mixtures thereof may be employed wherever required.

Also within the scope of this disclosure is a product containing micronized resin-therapeutic agent complex. In some embodiments, the product can be in packs in a form ready for administration, e.g., a blister pack, a bottle, syringes, foil packs, pouches, or other suitable containers. In some embodiments, the pharmaceutical compositions are in concentrated form in packs, optionally with the diluent required to make a final solution for administration. In some embodiments, the product contains a compound useful in the invention in solid form and, optionally, a separate container with a suitable suspension base or other carrier for the resin-therapeutic agent complex.

In some embodiments, the above packs/kits include other components, e.g., a metered dose apparatus/device, instructions for dilution, mixing and/or administration of the product, other containers, nasogastric tubes, etc. Other pack/kit components will be readily apparent to one of ordinary skill in the art. For example, various liquid metering devices for squeezable bottles have been described (U.S. Pat. Nos. 6,997,358, 3,146,919, filed in 1960, U.S. Pat. No. 3,567,079, filed in 1968, and in GB 2201395, filed in 1986). A device for dispensing multiple compositions is provided in U.S. Pat. No. 6,997,219.

The formulation can be administered to any patient in need thereof. Although preferred patients are human, animals, especially domestic animals such as dogs, cats, horses, cattle, sheep, goats, and fowl, may also be treated with the formulation. The amount of the active ingredients to be administered is chosen based on the amount which provides the desired dose to the patient in need of such treatment to alleviate symptoms or treat a condition.

### II. Methods of Preparation

The disclosed resin-therapeutic agent complex can be produced by first reducing an ion exchange resin to micronized size, prior to binding with a drug or drugs. It can also be produced by first binding resin with a drug or drugs and then reduced to a proper micronized size.

In one aspect, this disclosure also provides a method for preparing a pharmaceutical composition for oral administration. The method comprises: (1) micronizing ion-exchange resin particles by subjecting a suspension comprising ion-exchange resin particles having particle sizes larger than 50 μm to a size reduction process one or more times to obtain micronized ion-exchange resin particles having particle sizes equal to or less than 50 μm; (2) contacting the resulting micronized ion-exchange resin particles with at least one therapeutic agent to form resin-therapeutic agent complexes; and (3) admixing with a pharmaceutically acceptable carrier to form a pharmaceutical composition that when formulated into liquid or solid oral dosage forms can produce uniform dispersion of the resin-therapeutic agent complexes with less than 10 wt % (e.g., less than 8 wt %, less than 6 wt %, less than 5 wt %, less than 4 wt %, less than 3 wt %, less than 2 wt %, less than 1 wt %, less than 0.5 wt %) of the resin particles are in the form of aggregates.

In another aspect, the method comprises: (1) contacting ion-exchange resin particles having particle sizes larger than 50 μm with at least one therapeutic agent to form resin-therapeutic agent complexes; (2) micronizing the resin-therapeutic agent complexes by subjecting the resin-therapeutic agent complexes to a size reduction process one or more times to obtain micronized resin-therapeutic agent complexes having particle sizes equal to or less than 50 μm; and (3) admixing with a pharmaceutically acceptable carrier to yield a pharmaceutical composition that can be formulated into liquid or solid oral dosage forms that provide uniform dispersion of the resin-therapeutic agent complexes with less than 10 wt % (e.g., less than 8 wt %, less than 6 wt %, less than 5 wt %, less than 4 wt %, less than 3 wt %, less than 2 wt %, less than 1 wt %, less than 0.5 wt %) of the resin particles are in the form of aggregates.

There are many ways known in the art for the size reduction of ion-exchange resin or resin-therapeutic agent complexes. Milling is the most common way for size reduction. Milling may be conducted in its dry state (dry milling) or suspended in a liquid medium (wet milling). Some typical milling methods are described in greater detail below.

Jet milling is a dry milling process does not use mechanical components, but instead uses pressurized gas to create high particle velocity and high-energy impact between particles. In this micronization method, high velocity compressed air streams are injected into a chamber where the starting raw materials are fed by a rate controlled feeder. As the particles enter the air stream, they are accelerated and caused to collide with each other and the wall of the milling chamber with high velocities. Particle size reduction is brought about by a combination of impact and attrition. Impacts arise from collisions between the rapidly moving particles and particles onto the wall of the milling chamber. Attrition occurs at surfaces of particles as they move rapidly against each other, resulting in shear forces that may break them up. A classifier may be integrated into the milling system such that only particles that are sufficiently fine or have acquired dimensions below the predefined cut-off size are entrained in the exhausting air stream and removed from the milling chamber. After exiting the jet-mill chamber, the process gas is separated from the solid particles with a cyclone filter.

Media milling can be considered a modernized version of the ball mill. It is a classical wet milling technique wherein a sufficiently concentrated dispersion of drug particles in an aqueous or non-aqueous liquid medium is subjected to a traditional ball milling operation. The liquid medium prevents adhesion and subsequent compaction of the milled drug particles on the wall of the vessel and/or the surfaces of the milling balls, which is a common occurrence when the drug is milled in its dry state. This improves the yield of particles. The liquid may also serve additional purposes such as lubrication and coating of the newly-formed particle surfaces through various physicochemical interactions like electrostatic and hydrophobic interactions.

In media milling, mechanical attrition and impaction of the suspended resin/resinate particles are brought about by grinding balls, often termed as the milling media, constructed out of a variety of material such as glass (yttrium-stabilized), zirconium oxide, ceramics or highly cross-linked polystyrene resins. Pearl balls and beads are commonly used as well, in which case the techniques are termed pearl and bead milling, respectively. Unlike ball milling where the whole vessel rotates or oscillates/vibrates while in operation, the vessel remains stationary in media milling. Movement of the balls is initiated by a stirring or agitating device, often represented by several discs mounted on a central shaft rotating at high velocities, 20,000 rpm and above, within the vessel. Media milling is a continuous process wherein the drug suspension is pumped through the milling chamber to effect size reduction of the suspended material. Prior to their exit from the milling chamber, the milled particles pass through a screen that serves to separate the suspended, milled particles from the milling media.

Micronfluidizer milling uses wet mechanical milling to obtain micronized particles. Resinate or resin is suspended in a liquid medium and input into a reservoir that supports high solid content. A high-pressure pump generates forces up to 40,000 psi (2578 bar) to force the product stream into precisely engineered microchannels within the unique interaction chamber. Once inside the chamber, the product is exposed to consistent and intense impact and shear forces. This repeatable process results in tiny particles with a uniform distribution.

The micro or nanoparticles produced from milling possess a large surface/interfacial area, increased free energy, and decreased thermodynamic stability. These factors promote particle agglomeration. Mechanochemically-activated particle surfaces and amorphous regions generated during milling also increase the surface free energy of the particles, favoring agglomeration. Hence, when milling resonates/resin are co-milled together with certain adjuvants to minimize the conditions promoting agglomeration. These adjuvants are inert, non-toxic pharmaceutical excipients that function as a carrier and/or stabilizer of the drug in the milled product. Typically, the excipient employed is hydrophilic in nature and examples are gelatin, hydrophilic polymers such as polyvinylpyrrolidone, cellulose ethers, polyethylene glycol, polyvinyl alcohol or poloxamers; surfactants, etc.

Micronized resin/resinate can also be produced by using high pressure or piston-gap homogenization technique. Resinate/resin is suspended in a media solution that serves a carrier, lubricant, and suspending agent. Size reduction of resinate/resin is achieved by repeatedly cycling, with the aid of a piston, the resin/resinate suspension through a very small orifice at high velocity and pressure. The dimension of the orifice may be adjusted according to the size reduction requirement and also viscosity of the suspension and the applied pressure. When the suspension is forced through the gap at a high flow rate, the static pressure exerted on the liquid falls below the vapor pressure of the liquid at the prevailing temperature (Bernoulli's equation). As a result, the liquid boils, and gas bubbles are formed which collapse when the liquid exits from the gap and normal pressure are resumed. The powerful cavitation forces arising from the formation and collapse of the gas bubbles, coupled with a shearing effect, bring about reduction of the material. The extent of subdivision of the resinate/resin depends on the pressure applied as well as the number of passes or homogenization cycles the resinate suspension is subjected to during the process.

Adsorption of the pharmaceutically active agent onto the ion exchange resin particles to form the pharmaceutically active agent/resin complex is a well-known technique, as shown in U.S. Pat. Nos. 2,990,332 and 4,221,778. In general, the pharmaceutically active agent is mixed with an aqueous suspension of the resin for a certain period of time. Adsorption of pharmaceutically active agents onto the resin may be detected by measuring a change in the pH of the reaction medium, or by measuring a change in concentration of pharmaceutically active agents.

Binding of pharmaceutically active agent to resin can be accomplished according to four general reactions. In the case of a basic pharmaceutically active agent, these are: (a) resin (Na-form) plus pharmaceutically active agent (salt form); (b) resin (Na-form) plus pharmaceutically active agent (as free base); (c) resin (H-form) plus pharmaceutically active agent (salt form); and (d) resin (H-form) plus pharmaceutically active agent (as free base). All of these reactions except (d) have cationic by-products, by competing with the cationic pharmaceutically active agent for binding sites on the resin, reduce the amount of pharmaceutically active agent bound at equilibrium. For basic pharmaceutically active agents, stoichiometric binding of pharmaceutically active agent to resin is accomplished only through reaction (d).

Four analogous binding reactions can be carried out for binding an acidic pharmaceutically active agent to an anion exchange resin. These are: (a) resin (Cl-form) plus pharmaceutically active agent (salt form); (b) resin (Cl-form) plus pharmaceutically active agent (as free acid); (c) resin (OH-form) plus pharmaceutically active agent (salt form); and (d) resin (OH-form) plus pharmaceutically active agent (as free acid). All of these reactions except (d) have ionic by-products and the anions generated when the reactions occur compete with the anionic pharmaceutically active agent for binding sites on the resin with the result that reduced levels of pharmaceutically active agent are bound at equilibrium. For acidic pharmaceutically active agents, stoichiometric binding of pharmaceutically active agent to resin is accomplished only through reaction (d).

For preparing the complexes, the batch equilibration is the preferred practice when loading a drug into finely divided ion exchange resin powders. Due to its fine particle size, micronized ion exchange resin does not lend itself to conventional columnar operations used with ion exchange resins. The total ion exchange capacity represents the maximum achievable capacity for exchanging cations or anions measured under ideal laboratory conditions. The capacity which will be realized when loading a drug onto ion exchange resin will be influenced by such factors as the inherent selectivity of the ion exchange resin for the drug, the drug's concentration in the loading solution and the concentration of competing ions also present in the loading solution. The rate of loading will be affected by the activity of the drug and its molecular dimensions as well as the extent to which the polymer phase is swollen during loading.

When utilizing a batch or equilibrium process for loading a drug onto an ion exchange resin, it may be desirable to load as much as possible of the substance of value onto the ion exchange resin. Complete transfer of the drug from the loading solution is not likely in a single equilibrium stage. Accordingly, more than one equilibration may be required in order to achieve the desired loading onto the ion exchange resin. The use of two or more loading stages, separating the resin from the liquid phase between stages, is a means of achieving maximum loading of the drug onto the ion exchange resin although the loss of drug from the liquid phase of the final stage occurs.

The amount of drug that can be loaded onto a resin will typically range from about 1% to about 90% by weight of the drug-ion exchange resin particles. A skilled artisan with limited experimentation can determine the optimum loading for any drug resin complex. In one embodiment, loading of about 0.00001% to about 80% by weight, about 0.0001% to about 70% by weight, about 0.001% to about 60% by weight, about 0.01% to about 50% by weight, or about 0.1% to about 40% by weight, of the drug-ion exchange resin particles can be employed.

To prepare the resin-therapeutic agent complexes for this invention, the ion-exchange resin particles and the therapeutic agent can be mixed at a ratio of the therapeutic agent to the resin particle is in the range from 0.000000:1 to 3:1 by weight, calculated on a moisture-free, free acid or base basis. In some embodiments, the ratio of the therapeutic agent to the resin particle is in the range from 0.00000:1 to 1:1 by weight. In some embodiments, the ratio of the therapeutic agent to the resin particle is in the range from 0.0000:1 to 0.8:1 by weight. In some embodiments, the ratio of the therapeutic agent to the resin particle is in the range from 0.000:1 to 0.6:1 by weight. In some embodiments, the ratio of the therapeutic agent to the resin particle is in the range from 0.001:1 to 0.4:1 by weight.

The resin-therapeutic complex thus formed can be collected by filtration and washed with appropriate solvents to remove any unbound drug or by-products. The complexes can be air-dried in trays, in a fluid bed dryer, or other suitable dryers, at room temperature or at elevated temperature. In some embodiments, the formed resinates, either in suspension form right after the complexation process or collected and dried as an intermediate powder, can be further processed into other common drug dosage forms.

In one embodiment, the ratio of pharmaceutically active agent and resin should be low enough to achieve a high percentage of adsorption (>90%), so the washing and removal of any unadsorbed active is not necessary for avoiding the unpleasant taste of the free drug. This will avoid the process steps of collecting and drying the micronized resinate to reduce manufacturing time and costs.

In another embodiment, when a high percentage of adsorption cannot be achieved, the micronized resinate is collected and washed with water or an organic solvent to removal unadsorbed pharmaceutically active agent. The washed resinate is then collected. The collection can be achieved by filtration followed by tray drying at room or elevated temperature or by spray drying with or without additional additives. The washed resinate can also be collected by a freeze-drying process.

In some embodiments, after the complexation of resin and therapeutic agents is completed, the resin-therapeutic agent complexes are collected and dried, resulted in a dry powder blend of the resin-therapeutic agent complexes. The resulting dry powder blend of the resin-therapeutic agent complexed can be resuspended and re-dispersed in an liquid phase (e.g., water, organic solvent) to prepare a liquid suspension. In some embodiments, one or more dispersing agents can be added to the resin and therapeutic agent mixture before the collection and drying process. The addition of dispersing agent reduces the aggregation of resin or the resin-therapeutic agent complexes during the collection and drying process and during the resuspension process of the dry power blend of the resin-therapeutic complexes.

The micronized resinate, when collected and dried without any other additives, can form bigger particle size agglomerates in the process. These agglomerates often times cannot be easily dispersed back to the primary micronized resinate. These bigger particle size agglomerates can cause grittiness in final drug dosage forms and should be eliminated. Dispersion aids are materials that aid in breaking up these agglomerates when being resuspended in a liquid medium or further processed into other common drug dosage forms. They can be used alone or in combination. They can be added into the solution that suspends the micronized resinate and dried together with the micronized resinate.

In some embodiments, the methods described above further comprise adding a dispersing agent before or after the step of micronizing, thereby when the pharmaceutical composition is re-dispersed in a liquid medium, uniform dispersion of the resin-therapeutic agent complexes is produced and less than 10 wt % (e.g., less than 8 wt %, less than 6 wt %, less than 5 wt %, less than 4 wt %, less than 3 wt %, less than 2 wt %, less than 1 wt %, less than 0.5 wt %) of the resin particles are in the form of aggregates.

In some embodiments, the dispersing agent is: (1) a water-soluble substance selected from the group consisting of water-soluble polymers, hydrophilic surfactants, sugars, such as gelatin, Arabic gum, agarose, polyvinyl pyrrolidone, hydroxypropyl methylcellulose, hydroxypropyl cellulose, sodium carboxymethyl cellulose, mannitol, lactose, sucrose, sodium lauryl sulfate, poloxamers, or combinations thereof, or (2) a solvent-soluble material selected from the group consisting of solvent-soluble polymers, lipophilic surfactants, phospholipids, fatty acids, such as polyvinyl pyrrolidone, phosphatidylcholine, phosphatidylethanolamine, stearate acid, oleic acid, or combinations thereof.

The resin-therapeutic agent complexes with and without dispersion aids can be further processed, together with suitable excipients (binder, filler, disintegrant, glidant, lubricant, coating, colorant, flavors, etc.), and using suitable manufacturing processes (blending, granulation, compression, coating, Wurster coating, extrusion-spheronization etc.), into various type of tablet dosage forms known in the art, such as immediate-release tablets, extended-release tablets, chewable tablets, ODT tablets, mini-tablets, and orally disintegrating mini-tablets.

For example, the resin-therapeutic agent complexes can be further processed, together with suitable excipients, and using suitable manufacturing processes, into oral film/strip dosage forms known in the art, such as oral soluble films, ODT films. The resin-therapeutic agent complexes can also be further processed, together with suitable excipients, and using suitable manufacturing processes, into various types of oral liquid dosage forms known in the art, such as oral suspension, reconstitution powder for oral suspension.

The compositions may be formulated using known carriers or excipients, using well-established techniques. Without being limited thereto, such conventional carriers or excipients include diluents (i.e. lactose, mannitol, dicalcium phosphate, microcrystalline cellulose, hydroxypropyl methylcellulose (HPMC), etc.), granulating agents (water, ethanol, isopropanol etc.) binders and adhesives (i.e. starch paste, gums, polyvinyl pyrrolidone, Carboxymethylcellulose, HPMC and acrylic derivatives), disintegrants (starch, sodium starch glycolate, croscarmellose, crospovidone, etc.) lubricants (i.e., magnesium stearate, calcium stearate, vegetable oils, polyethylene glycols, talc, sodium acetate, sodium lauryl sulphate, polyoxyethylene monostearate), solubilizers and humectants (Tweens, Spans, propylene glycol, glycerol, etc.), buffers (acetate, citrate, phosphate, Tris, etc.) and adsorbents (colloidal silica [Aerosil], dicalcium phosphate, magnesium oxide, calcium carbonate, etc.). Colorants, flavors, preservatives viz. Methyl, ethyl and propyl parabens, sodium benzoate, potassium sorbate, sweeteners viz. Aspartame, saccharin and its salts, etc. and viscosity enhancers viz. cellulose, gums, carbopol, etc. and above-mentioned excipients are employed to prepare a particular medicated composition.

Reconstituted suspensions from dry formulations may be obtained by dispersing the drug/resin compositions in a suitable aqueous vehicle, optionally with the addition of suitable viscosity-enhancing agent(s) (e.g., cellulose derivatives, guar gum, xanthan gum, etc.). Non-aqueous suspensions may be obtained by dispersing the drug/resin compositions in a suitable non-aqueous based vehicle, optionally with the addition of suitable viscosity-enhancing agent(s) (e.g., hydrogenated edible fats, aluminum stearate, etc.). Suitable non-aqueous vehicles include, for example, almond oil, Arachis oil, soybean oil or fractionated vegetable oils such as fractionated coconut oil. The drug resinates may also be supplied in a ready mixed composition in an aqueous base as pleasantly flavored suspensions.

### III. Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The terms “resin-drug therapeutic complex,” “drug resinate,” “drug-resin complex,” “drug-resin conjugate,” are used interchangeably in this disclosure.

The terms “decrease,” “reduced,” “reduction,” “decrease,” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, the term “pharmaceutical grade” means that certain specified biologically active and/or inactive components in the drug must be within certain specified absolute and/or relative concentration, purity and/or toxicity limits and/or that the components must exhibit certain activity levels, as measured by a given bioactivity assay. Further, a “pharmaceutical grade compound” includes any active or inactive drug, biologic or reagent, for which a chemical purity standard has been established by a recognized national or regional pharmacopeia (e.g., the U.S. Pharmacopeia (USP), British Pharmacopeia (BP), National Formulary (NF), European Pharmacopoeia (EP), Japanese Pharmacopeia (JP), etc.). Pharmaceutical grade further incorporates suitability for administration by means including topical, ocular, parenteral, nasal, pulmonary tract, mucosal, vaginal, rectal, intravenous, and the like.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### IV. Examples

### Example 1: Preparation of Micronized Cation Exchange Resin with Carboxylic Acid Groups and has a Particle Size 100%<50 μm or Preferably <30 μm

32 g of Amberlite IRP 64 (with carboxylic acid groups) was added to 800 ml of purified water. The suspension was processed using a Microfluidizer (Microfluidics model M-110P) with the following parameters.

Pressure: 25,000 psi

Chamber orifice diameter: 200 μm and 87 μm in serial connection

Time of cycling: 150 minutes

The maximum loop temperature was maintained <18° C. using an ice bath.

The resulted suspension was observed under the microscope. The morphology and particle size are demonstrated in Picture 1. No particles are bigger than 50 μm in size of any dimension.

The particle size distribution of the Amberlite IRP64 resin before and after micronization was measured using a Malvern Mastersizer 3000 particle sizer. The result is summarized in the table below.

| | Particle Size (μm) | | |
|---|---|---|---|
| Sample | D(90) | D(50) | D(10) |
| Amberlite IRP64 | 151 | 75 | 17 |
| Before Micronization | | | |
| Amberlite IRP64 | 17.3 | 9.6 | 4.5 |
| After Micronization | | | |

### Example 2: Preparation of Micronized Cation Exchange Resin with Sulfonic Acid Groups and has a Particle Size 100%<50 μm or Preferably <30 μm

32 g of Amberlite IRP 69 (with sulfonic acid groups) was added to 800 ml of purified water. The suspension was processed using a Microfluidizer (Microfluidics model M-110P) with the following parameters.

Pressure: 25,000 psi

Chamber orifice diameter: 200 μm and 87 μm in serial connection

Time of cycling: 120 minutes

The maximum loop temperature was maintained <20° C. using an ice bath.

The resulted suspension was observed under the microscope. The morphology and particle size are demonstrated in Picture 2. No particles are bigger than 50 μm in size of any dimension.

The particle size distribution of the Amberlite IRP64 resin before and after micronization was measured using a Malvern Mastersizer 3000 particle sizer. The result is summarized in the table below.

| | Particle Size (μm) | | |
|---|---|---|---|
| Sample | D(90) | D(50) | D(10) |
| Amberlite IRP69 | 132 | 67 | 21 |
| Before Micronization | | | |
| Amberlite IRP69 | 7.7 | 4.2 | 2.3 |
| After Micronization | | | |

### Example 3: Preparation of Micronized Anion Exchange Resin with Cholestyramine Groups and has a Particle Size 100%<50 μm or Preferably <30 μm

2 g of Duolite AP143 (with cholestyramine groups) was added to 200 ml of purified water. The suspension was processed using a Microfluidizer (Microfluidics model M-110P) with the following parameters.

Pressure: 20,000 psi

Chamber orifice diameter: 200 μm and 87 μm in serial connection

Time of cycling: 60 minutes

The maximum loop temperature was maintained <20° C. using an ice bath.

The resulted suspension was observed under the microscope. The morphology and particle size are demonstrated in Picture 3. No particles are bigger than 50 μm in size of any dimension. The particle size distribution of the Duolite AP143 resin before and after micronization was measured using a Malvern Mastersizer 3000 particle sizer. The result is summarized in the table below.

| | Particle Size (μm) | | |
|---|---|---|---|
| Sample | D(90) | D(50) | D(10) |
| Duolite AP143 | 100 | 37 | 6 |
| Before Micronization | | | |
| Duolite AP143 | 3.2 | 0.08 | 0.02 |
| After Micronization | | | |

### Example 4: Preparation of Freeze-Dried Micronized Resin-Therapeutic Agent Complexes Using Micronized Resins that have Particle Size 100%<50 μm or Preferably <30 μm

Micronized resin-therapeutic agent complexes were prepared using the following ion-exchange resin and active compositions and procedures.

| Batch # | 9055-10 | 9055-11 | 9055-09 | 9055-08 |
|---|---|---|---|---|
| Guanfacine HCl | 4 mg | — | — | — |
| Oseltamivir Phosphate | — | 6 mg | — | — |
| Pramipexole HCl | — | — | 0.25 mg | — |
| Ropinirole HCl | — | — | — | 0.25 mg |
| Micronized Amberlite IRP64 | 18.37 mg | — | 4.75 mg | 4.75 mg |
| Micronized Amberlite IRP69 | — | 50.66 mg | — | — |

### Sample Preparation Procedures:

Micronized ion-exchange resins (Amberlite IRP64 and IRP 69) were prepared following procedures disclosed in Examples 1 & 2. The micronized ion-exchange resins were complexed with the actives in purified water. The pH of the ion-exchange resin-active suspension was adjusted as necessary to improve complexation efficiency. The ion-exchange resin-active suspensions were frozen using liquid nitrogen, then freeze-dried using a Labconco freeze dryer (FreeZone 2.5) operating at a 0.22-0.28 mbar vacuum pressure and −49° C. temperature for 18-20 hours.

### Example 5: Preparation of Freeze-Dried Compositions of Micronized Resin-Therapeutic Agent Complexes with Dispersing Aids Using Micronized Resins that have Particle Size 100%<50 μm or Preferably <30 μm

Micronized resin-therapeutic agent complexes with dispersing aids were prepared according to the following unit compositions and preparation procedure.

| Dry Suspension Batch # | 9053-82 | 9055-02 | 9053-76 | 9053-78 |
|---|---|---|---|---|
| Guanfacine HCl | 4 mg | — | — | — |
| Oseltamivir Phosphate | — | 6 mg | — | — |
| Pramipexole HCl | — | — | 0.25 mg | — |
| Ropinirole HCl | — | — | — | 0.25 mg |
| Micronized | 18.37 mg | — | 4.75 mg | 4.75 mg |
| Amberlite IRP64 | | | | |
| Micronized | — | 50.66 mg | — | — |
| Amberlite IRP69 | | | | |
| Poloxamer 188 | 4.59 mg | 12.67 mg | 1.19 mg | 1.19 mg |
| Sodium Alginate | 0.46 mg | 1.27 mg | 0.12 mg | 0.12 mg |
| (CR8223) | | | | |
| Mannitol | 13.77 mg | 38 mg | 3.56 mg | 3.56 mg |

### Sample Preparation:

Micronized ion-exchange resins (Amberlite IRP64 and IRP 69) were prepared following procedures disclosed in Examples 1-2. The micronized ion-exchange resins were complexed with the actives in purified water under stirring to obtain micronized ion-exchange resin-active complexes. The pH of the ion-exchange resin-active suspension was adjusted as necessary to improve complexation efficiency. At the end of the complexation step, add sodium alginate, mannitol, and Poloxamer 188 into the ion-exchange resin-active suspension under stirring. Continue mixing until sodium alginate, mannitol, and Poloxamer 188 were dissolved. Transfer the solutions into freeze-drying glassware and freeze them using liquid nitrogen. The resulted frozen mixture is freeze-dried using a Labconco freeze dryer (FreeZone 2.5) operating at a 0.11-0.28 mbar vacuum pressure and −48-50° C. for 18-20 hours.

### Example 6: Preparation of Freeze-Dried Compositions of Un-Micronized Resin-Therapeutic Agent Complexes with Dispersing Aids Using Un-Micronized Commercial Resins

A. Preparation of Un-Micronized Resin-Therapeutic Agent Complexes.

Un-micronized resin-therapeutic agent complexes are prepared using the following ion-exchange resin and active compositions and procedures.

| Batch # | 9053-88 | 9053-87 | 9053-92 | 9055-01 |
|---|---|---|---|---|
| Guanfacine HCl | 4 mg | — | — | — |
| Oseltamivir | — | 6 mg | — | — |
| Phosphate | | | | |
| Pramipexole HCl | — | — | 0.25 mg | — |
| Ropinirole HCl | — | — | — | 0.25 mg |
| Amberlite IRP64 | 18.37 mg | — | 4.75 mg | 4.75 mg |
| Amberlite IRP69 | — | 50.66 mg | — | — |

### Sample Preparation Procedures:

In a container, add 100 mL of purified water. Weigh 4 g of commercial ion-exchange resins (Amberlite IRP64 or IRP 69) and suspend in the water. Weigh the actives and add to the resin suspensions under stirring. The pH of the ion-exchange resin-active suspension was adjusted as necessary to improve complexation efficiency. The endpoint of the complexation was determined by measuring the free actives in water using UV. The complexation efficiency was calculated and summarized in the table below.

| | Complexation |
|---|---|
| Name of Resin-Active Complex | Efficiency |
| Amberlite IRP64-Guanfacine complex | 99.3% |
| Amberlite IRP69-Oseltamivir complex | 96.7% |
| Amberlite IRP64-Pramipexole complex | 99.5% |
| Amberlite IRP64-Ropinirole complex | 98.8% |

B. Preparation of Dry Compositions Containing Un-Micronized Resin-Therapeutic Agent Complexes and Dispersing Aids Using the Freeze-Drying Process.

Dispersing aids, i.e., poloxamer 188, sodium alginate, and mannitol, were weighed according to the following composition and added to the resulted suspensions from Step A of this example. The mixtures were stirred until the dispersing aids were dissolved.

| Dry Composition Batch # | 9053-90 | 9053-91 | 9055-03 | 9055-04 |
|---|---|---|---|---|
| Amberlite IRP64-Guanfacine | 22.37 | — | — | — |
| complex | | | | |
| Amberlite IRP69-Oseltamivir | — | 56.66 | — | — |
| complex | | | | |
| Amberlite IRP64-Pramipexole | — | — | 5.00 | — |
| complex | | | | |
| Amberlite IRP64-Ropinirole | — | — | — | 5.00 |
| complex | | | | |
| Poloxamer 188 | 4.59 mg | 12.67 mg | 1.19 mg | 1.19 mg |
| Sodium Alginate (CR8223) | 0.46 mg | 1.27 mg | 0.12 mg | 0.12 mg |
| Mannitol | 13.77 mg | 38.00 mg | 3.56 mg | 3.56 mg |

The resulting mixtures were freeze-dried using a Labconco freeze dryer (FreeZone 2.5) operating at a 0.11-0.21 mbar vacuum pressure and −49-−50° C. for 18 hours.

### Example 7: Preparation of Oven-Dried Compositions of Micronized Resin-Therapeutic Agent Complexes Using Micronized Resins that have Particle Size 100%<50 μm or Preferably <30 μm (without Dispersing Aids)

Micronized resin-therapeutic agent complex suspensions were prepared using the same ion-exchange resin and active compositions and procedures as disclosed in Example 4 before the freeze-drying steps. A portion of these suspensions (50 mL) was filtered and the particles collected were dried in an oven at 50° C. for 20 hours. The following four samples were obtained.

| Sample Name | Batch # |
|---|---|
| Guanfacine/MR64/OD powder | 9055-47 |
| Oseltamivir/MR69/OD powder | 9055-15 |
| Pramipexole/MR64/OD powder | 9055-48 |
| Ropinirole/MR64/OD powder | 9055-49 |

MR64: micronized IRP64 resin; MR69: micronized IRP69 resin; OD: oven-dried

### Example 8: Preparation of Oven-Dried Compositions of Micronized Resin-Therapeutic Agent Complexes with Dispersing Aids Using Micronized Resins that have Particle Size 100%<50 μm or Preferably <30 μm

Dry powders prepared following the same formula composition and procedures as disclosed in Example 5 were used in the preparation of these samples. Each dry powder was placed in a container. The dry powder was granulated by using purified water under mixing to form wet granules. The granules were dried in an oven at 50° C. for 3 hours. The dried granules were passed through a 30 mesh stainless steel screen to form the final granules. The following four samples were obtained.

| Sample Name | Batch # |
|---|---|
| Guanfacine/MR64/DA/OD granules | 9055-16 |
| Oseltamivir/MR69/DA/OD granules | 9055-17 |
| Pramipexole/MR64/DA/OD granules | 9055-18 |
| Ropinirole/MR64/DA/OD granules | 9055-19 |

MR64: micronized IRP64 resin; DA: MR69: micronized IRP69 resin; dispersing aid; OD: oven dried

### Example 9: Demonstrate the Dispersibility (i.e., Elimination of Agglomerates) of Freeze-Dried Compositions of Micronized Resin-Therapeutic Agent Complexes with Dispersing Aids Using Micronized Resins that have Particle Size 100%<50 μm or Preferably <30 μm in Common Food Drinks, Such as Water, Milk, Soy Milk, and Infant Formula, Etc.)

This study demonstrated that the freeze-dried compositions of micronized resin-therapeutic agent complexes with dispersing aids using micronized resins were able to easily dispersed in common food drinks, such as water, milk, soy milk, and infant formula without agglomerates, and the dispersed micronized resin-therapeutic agent complexes return to their original particle size of 100%<50 μm or preferably <30 μm, so the mouthfeel of the common food drinks are not negatively affected by grittiness. The freeze-dried compositions (or dry suspension) used for this study were prepared in Example 5. An aliquot of powder of the dry suspension was added to a container. Water, milk, soy milk or infant formula, 5 ml, was added to the container to disperse the dry powder. The dispersion of the powders was observed visually. After 30 to 60 seconds, a sample of the dispersion was observed under a microscope. A microscopic picture was taken.

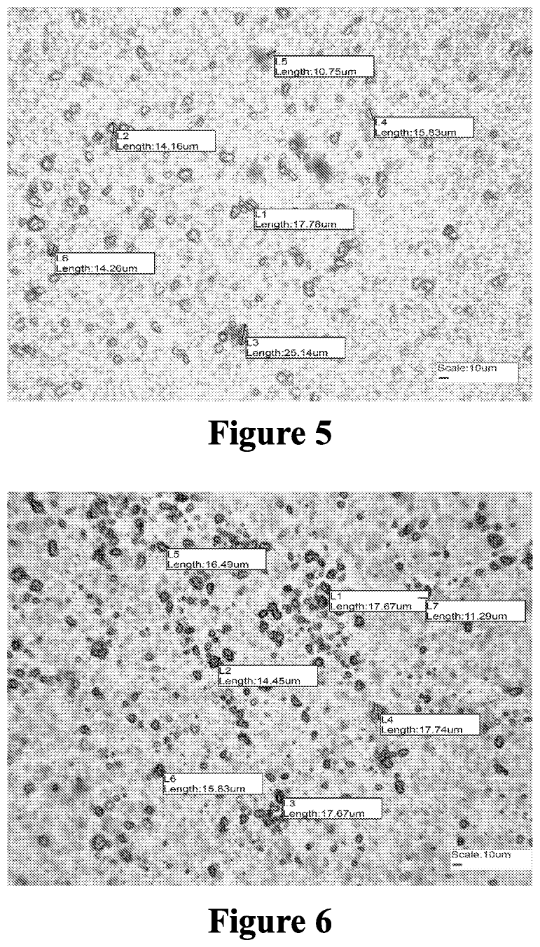

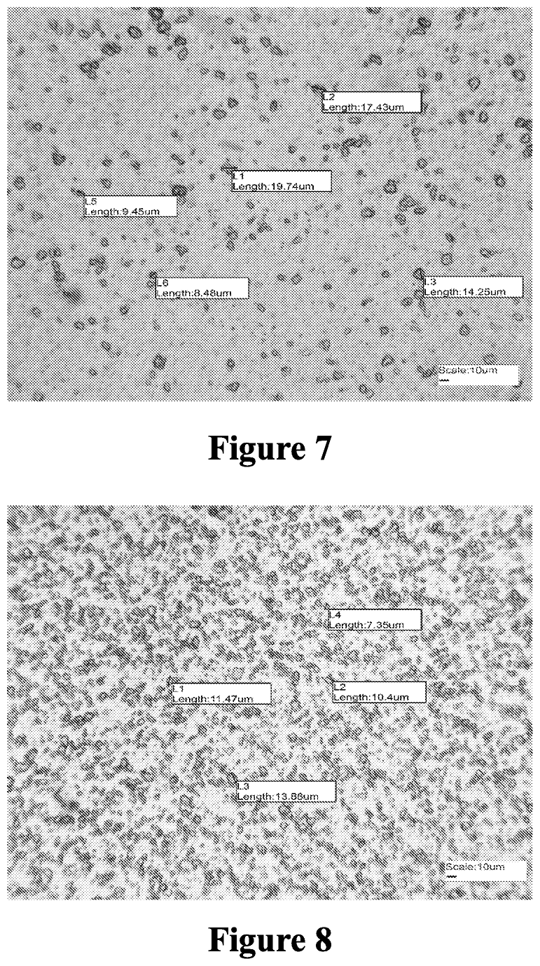

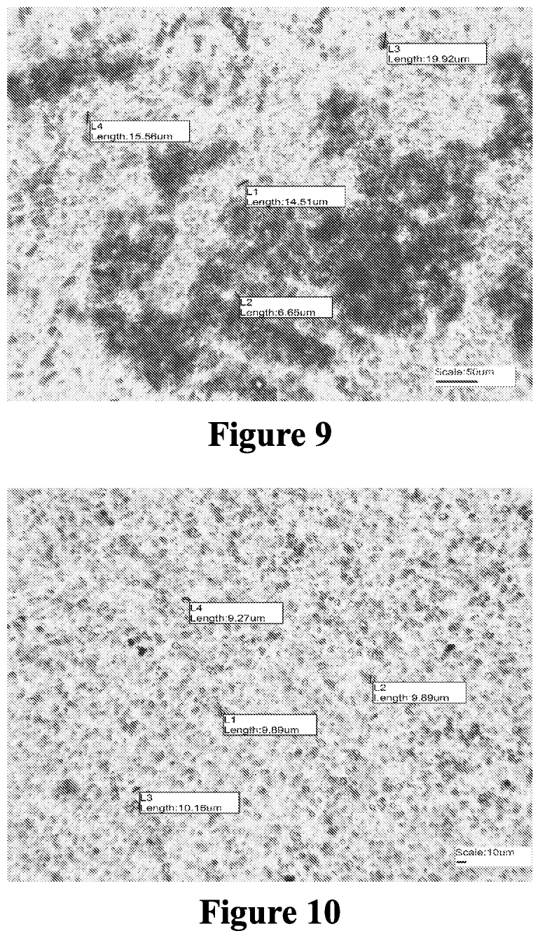

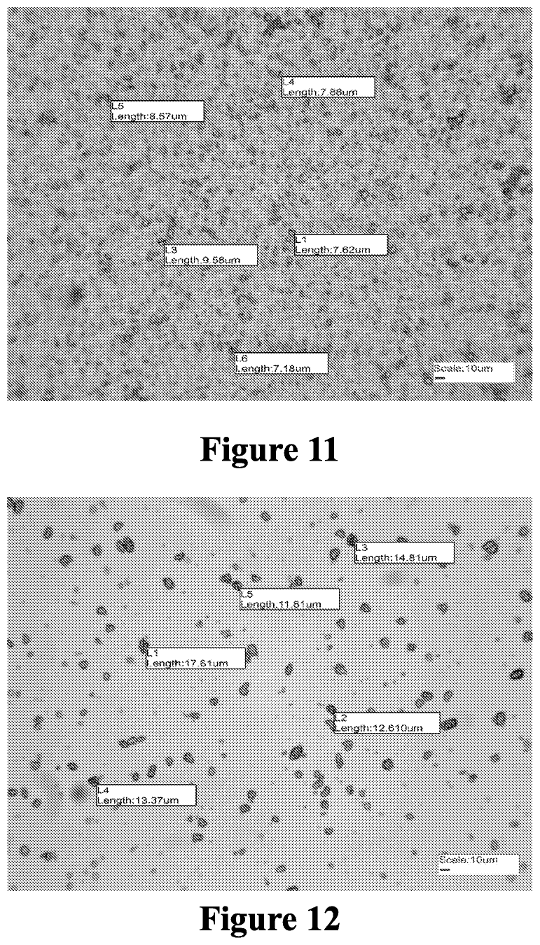

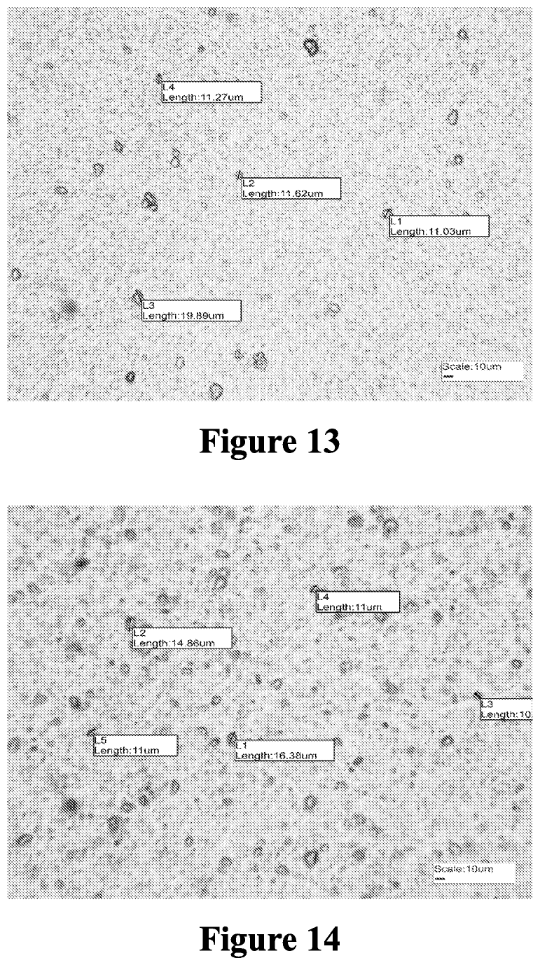

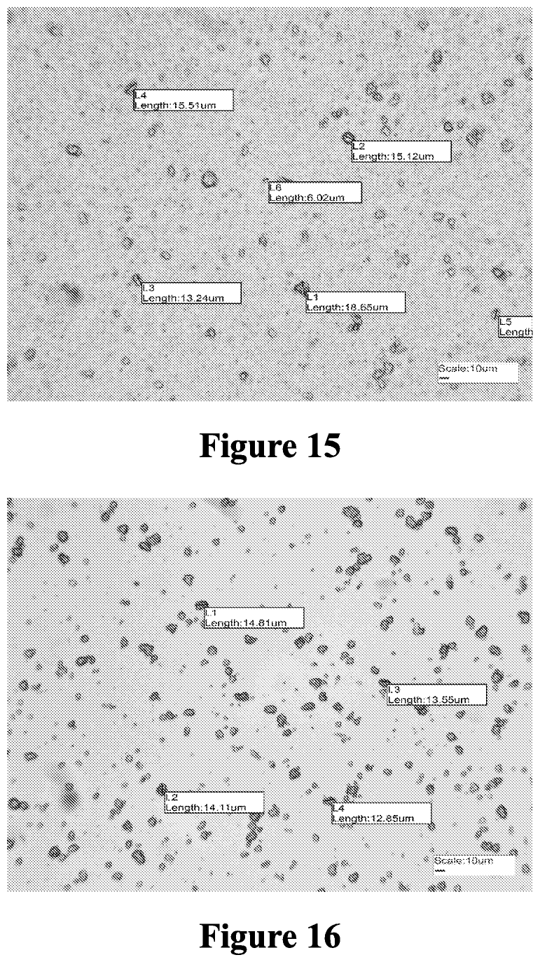

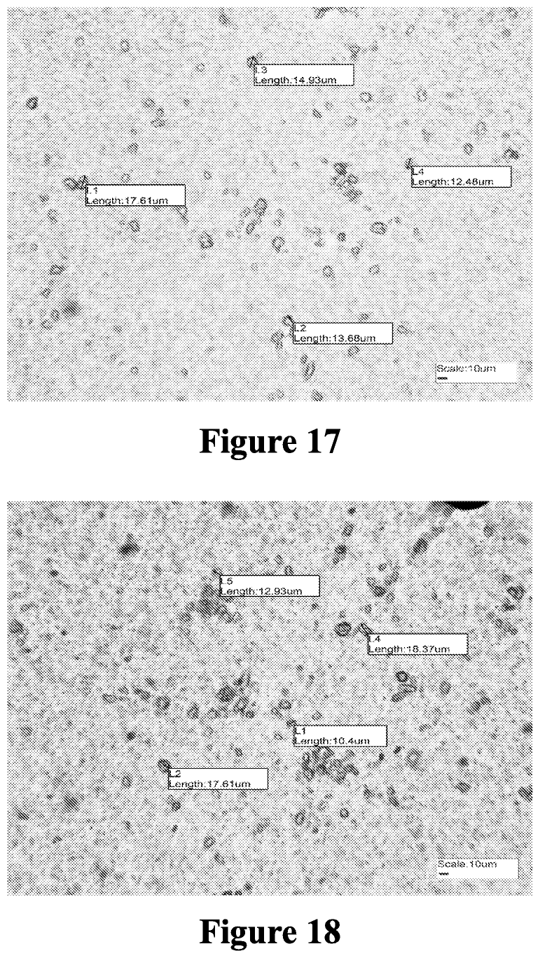

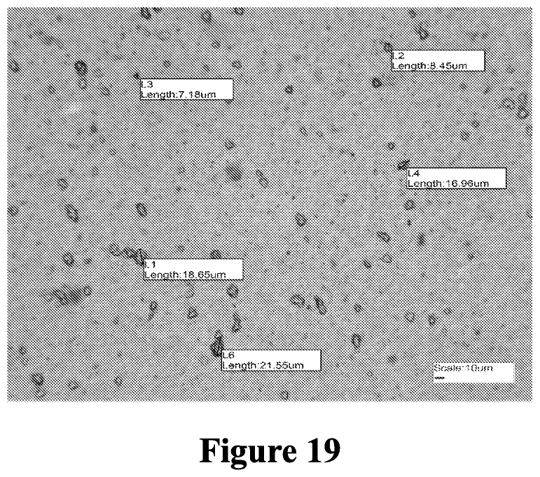

The results of this evaluation are summarized in the table below.

| | | | Description |
|---|---|---|---|
| | | Description of the | of the |
| | | Constitution Process | Constituted |
| Preparation | Dispersing | (e.g, ease of | Suspension |
| (Batch #) | Media | constitution) | (Pictures) |
| Dry suspensions | Water | Very easy, disperse | FIG. 4 |
| containing micronized | | totally less than 30 | |
| resin-Guanfacine | | seconds. | |
| complexes | Whole Milk | Easy, disperse totally | FIG. 5 |
| (9053-82) | | within one minute | |
| | Soy Milk | Easy, disperse totally | FIG. 6 |
| | | within one minute | |
| | Infant formula | Easy, disperse totally | FIG. 7 |
| | | within one minute | |
| Dry suspensions | Water | Very easy, disperse | FIG. 8 |
| containing micronized | | totally less than 30 | |
| resin-Oseltamivir | | seconds. | |
| complexes | Whole Milk | There are some | FIG. 9 |
| (9055-02) | | aggregates in one- | |
| | | minute dispersion | |
| | Soy Milk | Easy, disperse totally | FIG. 10 |
| | | within one minute | |
| | Infant formula | There are some | FIG. 11 |
| | | agglomerates in one- | |
| | | minute dispersion, | |
| | | disperse totally after | |
| | | slightly shaking. | |
| Dry suspensions | Water | Very easy, disperse | FIG. 12 |
| containing micronized | | totally less than 30 | |
| resin-Pramipexole | | seconds. | |
| complexes | Whole Milk | Easy, disperse totally | FIG. 13 |
| (9053-76) | | within one minute | |
| | Soy Milk | Easy, disperse totally | FIG. 14 |
| | | within one minute | |
| | Infant formula | Easy, disperse totally | FIG. 15 |
| | | within one minute | |
| Dry suspensions | Water | Very easy, disperse | FIG. 16 |
| containing micronized | | totally less than 30 | |
| resin-Ropinirole | | seconds. | |
| complexes | Whole Milk | Easy, disperse totally | FIG. 17 |
| (9053-78) | | within one minute | |
| | Soy Milk | Easy, disperse totally | FIG. 18 |
| | | within one minute | |
| | Infant formula | Easy, disperse totally | FIG. 19 |
| | | within one minute | |

The data demonstrated that the dry suspensions containing micronized resin-therapeutic active complexes are compatible with water and other common food drinks including infant formula. These dry suspensions containing micronized resin-therapeutic active complexes are also capable of quickly dispersing and returning to micronized primary complex particles size of 100%<50 μm or preferably <30 μm in these food drinks from the dry state.

### Example 10: Demonstrate the Mouthfeel Benefit of Suspensions Constituted from Dry Compositions Containing Micronized Ion-Exchange Resin-Active Complexes with Dispersion Aids Prepared in Examples 4-8

Dry compositions containing micronized ion-exchange resin-active complexes prepared in Examples 4-8 were evaluated for taste and mouthfeel using the following procedure. The taste and mouthfeel evaluation was conducted by a two-person panel. An active solution containing the same amount of the active as in each suspension is prepared as a reference of the pure active taste and for comparison purposes.

An aliquot of powder of a dry suspension was added to a container. Purified water USP, 5 ml is added to the container to constitute the powder into a suspension. Once the powder was fully wet and dispersed, approximately 1 ml sample of the suspension was drawn using a dropper. Drop the suspension sample on the tongue and immediately close the mouth to assess the taste and mouthfeel without swallowing. Spit the sample out and rinse mouth with purified water before evaluating the next sample.

The results are summarized in the table below.

Abbreviation:

R64: Amberlite IRP64 ion exchange resin (not micronized)

R69: Amberlite IRP69 ion exchange resin (not micronized)

MR64: micronized Amberlite IRP64 ion exchange resin

MR69: micronized Amberlite IRP69 ion exchange resin

DA: dispersing aid (sodium alginate and poloxamer 188)

FD: freeze-dried

OD: oven-dried

| | | | | |
|---|---|---|---|---|
| | | Dry | | |
| | | Sample | | |
| | Dose | Weight | | |
| Suspension Preparation | (mg) | (mg) | Taste | Mouthfeel |

Guanfacine

| | | | | |
|---|---|---|---|---|
| Guanfacine HCl solution | 2 | 2 | Very bitter | Feels like water |
| (9055-20-1) | | | | |
| Guanfacine/R64/DA/FD | 2 | 41.19 | Tasteless to | Slight grittiness |
| (9053-90) | | | chalky taste | to smooth |
| Guanfacine/MR64/OD | 2 | 22.37 | Slight resin to | Sandy feel |
| (9055-47) | | | resin taste | |
| Guanfacine/MR64/FD | 2 | 22.37 | Tasteless | Smooth |
| (9055-12) | | | | |
| Guanfacine/MR64/DA/OD | 2 | 41.19 | Tasteless to | Smooth |
| (9055-16) | | | resin taste | |
| Guanfacine/MR64/DA/FD | 2 | 41.19 | Slightly sweet | Smooth |
| (9053-82) | | | | |

Oseltamivir

| | | | | |
|---|---|---|---|---|
| Oseltamivir phosphate solution | 6 | 30 | Very bitter | Feels like water |
| (9025-20-2) | | | | |
| Oseltamivir/R69/DA/FD | 6 | 543 | Initial tasteless | Slightly gritty to |
| (9053-91) | | | to slightly sweet | gritty |
| | | | taste; slight | |
| | | | bitter after taste | |
| Oseltamivir/MR69/OD | 6 | 283.3 | Tasteless to | Slightly gritty |
| (9055-15) | | | very bitter | |
| Oseltamivir/MR69/FD | 6 | 283.3 | Initial tasteless | Smooth |
| (9055-11) | | | to resin taste, | |
| | | | then resin taste | |
| | | | to very bitter | |
| | | | after taste | |
| Oseltamivir/MR69/DA/OD | 6 | 543 | Initial sweet to | Smooth |
| (9055-17) | | | slightly sweet | |
| | | | taste, then resin | |
| | | | to resin bitter | |
| | | | taste | |
| Oseltamivir/MR69/DA/FD | 6 | 543 | Initial slightly | Smooth |
| (9055-02) | | | sweet to resin | |
| | | | taste, then very | |
| | | | slight bitter to | |
| | | | bitter after taste | |

Pramipexole

| | | | | |
|---|---|---|---|---|
| Pramipexole HCl solution | 0.25 | 0.25 | Tasteless | Feels water |
| (9055-20-3) | | | | |
| Pramipexole/R64/DA/FD | 0.25 | 9.87 | Tasteless to | No gritty to slight |
| (9055-03) | | | resin taste | gritty |
| Pramipexole/MR64/OD | 0.25 | 5 | Tasteless to | Felt large |
| (9055-48) | | | resin taste | particles, gritty, |
| | | | | and sandy |
| Pramipexole/MR64/FD | 0.25 | 5 | Tasteless | Feels like water |
| (9055-09) | | | | |
| Pramipexole/MR64/DA/OD | 0.25 | 9.87 | Tasteless to | Feels no grittiness |
| (9055-18) | | | resin taste | |
| Pramipexole/MR64/DA/FD | 0.25 | 9.87 | Tasteless | Feels like water or |
| (9053-76) | | | | solution |

Ropinirole

| | | | | |
|---|---|---|---|---|
| Ropinirole HCl solution | 0.25 | 0.25 | Tasteless | Feels like water |
| (9055-20-4) | | | | |
| Ropinirole/R64/DA/FD | 0.25 | 9.87 | Tasteless to | Gritty and sandy |
| (9055-04) | | | resin taste | |
| Ropinirole/MR64/OD | 0.25 | 5 | Tasteless to | Slightly sandiness |
| (9055-49) | | | resin taste | |
| Ropinirole/MR64/FD | 0.25 | 5 | Tasteless | Feels like water or |
| (9055-08) | | | | solution, no |
| | | | | grittiness |
| Ropinirole/MR64/DA/OD | 0.25 | 9.87 | Tasteless to | No grittiness to |
| (9055-19) | | | resin taste | slight grittiness |
| Ropinirole/MR64/DA/FD | 0.25 | 9.87 | Tasteless | Feels like water or |
| (9053-78) | | | | solution, no |
| | | | | grittiness |

The outcome of this evaluation indicates that suspensions comprising un-micronized resin-active complexes always show various degrees of grittiness mouthfeel (in the active/R64 or R69/DA/FD cases), even with dispersion aids added. In contrast, suspensions comprising micronized resin-active complexes, in general, show no such grittiness mouthfeel if, upon constitution, the dry suspension can be sufficiently dispersed with little agglomerates. The dispersing aid is necessary to assure no grittiness mouthfeel. Suspensions comprising no dispersing aids could not be sufficiently dispersed if they were oven-dried. Because any drying process (e.g., oven drying) that creates agglomerates of resin-active complexes, even for micronized resin-active complexes, will produce large particles that cause gritty mouthfeel. This invention demonstrates that suspensions comprising micronized resin-active complexes with dispersing aids produce no large particle agglomerates and therefore result in little to no grittiness mouthfeel in both freeze-dried and oven-dried dry suspension powders.

### Example 11: Preparation of Compression Blend, Comprising Micronized Amberlite IRP64 Resin-Guanfacine Complex Prepared Using Micronized Resins that have a Particle Size 100%<50 μm or Preferably <30 μm, for Compressing Orally Disintegrating Tablets and Mini Tablets

A. Preparation of Micronized Amberlite IRP64 Ion Exchange Resin.

Micronized Amberlite IRP64 ion exchange resin was prepared following a similar procedure as disclosed in Example 1. Amberlite IRP64 ion exchange resin was suspended in purified water. This suspension was processed using a Microfluidizer (Microfluidics model M-110P) with the following parameters.

Pressure: 25,000 psi

Chamber orifice diameter: 200 μm and 87 μm in serial connection

Time of cycling: 150 minutes

Maximum loop temperature was maintained <18° C. using ice bath.

B. Preparation of Micronized Amberlite IRP64 Resin-Guanfacine Complex.

The following procedure was used for preparing the micronized Amberlite IRP64 resin-Guanfacine complex.

1. The following procedure was used for preparing the micronized Amberlite IRP64 resin-Guanfacine complex.

1. In a proper size container, charge 2000 mL purified water.

2. Under stirring, add approximately 4 g Guanfacine HCL into the purified water.

3. Weigh 200 g Amberlite IRP64 and added to the solution. Adjust the pH of the mixture to 6.1 using a 10 N NaOH solution.

4. Repeat step 2 & 3 until 49.7 g of Guanfacine HCL was added to the mixture.

5. Final adjust the pH of the mixture to pH 7.0. The free Guanfacine HCL in water was measured and found to be 0.245 g. The complexation efficiency was calculated as 99.5%.

6. The resulted un-micronized Amberlite IRP64 resin-Guanfacine complex particles were collected by filtration, and dried in an oven at 40° C. until the loss on drying became constant. The loss on drying was measured using a moisture balance and found to be 14.3%.

C. Preparation of Compression Blend Comprising Micronized Amberlite IRP64 Resin-Guanfacine Complex.

A compression blend was prepared using the following composition and procedures.

### Blend Formula Composition

| | Weight | |
|---|---|---|
| Ingredient | (g) | % |
| Micronized Amberlite IRP64 resin-Guanfacine | 31.5 | 5.6 |
| complex prepared in Step B of this example | | |
| PROSOLV EASTtab | 472.5 | 84.4 |
| Mannitol (Pearlitol 160C) | 56 | 10 |
| Purified Water | * | * |
| Blend total | 560 | 100 |

*Removed during the process.

### Procedure:

1. Weigh each ingredient according to blend formula composition.

2. Suspend the micronized Amberlite IRP64 resin-Guanfacine complex in the purified water, mix to disperse all particles and make sure no agglomerates were present.

3. Charge a high shear granulator (Key International KG5) with PROSOLV EASTtab and Mannitol, mix for 5 minutes at 300 rpm paddle speed without a chopper.

4. Granulate the powder mixture in Step 3 by adding the micronized Amberlite IRP64 resin-Guanfacine complex suspension from Step 2 over 1 minute and continue mixing for a total of 4 minutes at 300 rpm paddle speed without chopper.

5. Discharge the mixture and dry in an oven at 50° C. until the loss on drying is 2.0%.

6. Mill the dried granules using a Quadro Comil (model 193) fitted with #610 screen and 2258 rpm rotor speed to obtain a free-flowing powder.

### Example 12 Preparation of Compression Blend Comprising Un-Micronized Amberlite IRP64 Resin-Guanfacine Complexes Prepared Using Commercial Ion Exchange Resins

A. Preparation of Un-Micronized Amberlite IRP64 Resin-Guanfacine Complex.

The following procedure was used for preparing the un-micronized Amberlite IRP64 resin-Guanfacine complex.

1. In a proper size container, charge 2000 mL purified water.

2. Under stirring, add approximately 4 g Guanfacine HCL into the purified water.

3. Weigh 200 g Amberlite IRP64 and added to the solution. Adjust the pH of the mixture to 6.1 using a 10 N NaOH solution.

4. Repeat step 2 & 3 until 49.7 g of Guanfacine HCL was added to the mixture.

5. Final adjust the pH of the mixture to pH 7.0. The free Guanfacine HCL in water was measured and found to be 0.48 g. The complexation efficiency was calculated as 99.03%.

6. The resulted micronized Amberlite IRP64 resin-Guanfacine complex particles were collected by filtration and dried in an oven at 40° C. until the loss on drying became constant. The loss on drying was measured using a moisture balance and found to be 15%.

B. Preparation of Compression Blend Comprising Un-Micronized Amberlite IRP64 Resin-Guanfacine Complex.

A compression blend was prepared using the following formula composition.

| | Weight | |
|---|---|---|
| Ingredient | (g) | % |
| Un-micronized Amberlite IRP64 resin- | 31.5 | 5.6 |
| Guanfacine complex prepared in Step A | | |
| of this example | | |
| PROSOLV EASTtab | 472.5 | 84.4 |
| Mannitol (Pearlitol 160C) | 56 | 10 |
| Purified Water | * | * |
| Blend total | 560 | 100 |

*Removed during the process.

The procedures used in this example are identical to procedures described in Example 11 Step C, except the granules were dried to loss on drying of 2.4%. A free-flowing powder blend was obtained.

### Example 13: Preparation of Compression Blend Comprising Active Guanfacine HCl Active Only (without Ion Exchange Resin)

A compression blend comprising active Guanfacine HCl was prepared according to the following formula composition.

| | Weight | |
|---|---|---|
| Ingredient | (g) | % |
| Guanfacine HCl, USP | 5.6 | 1 |
| PROSOLV EASTtab | 498.4 | 89 |
| Mannitol (Pearlitol 160C) | 56 | 10 |
| Purified Water | * | * |
| Blend total | 560 | 100 |

The procedures used in this example are identical to procedures described in Example 11 Step C, except the granules, were dried to loss on drying of 2.3%. A free-flowing powder blend was obtained.

### Example 14: Preparation of Orally Disintegrating Tablet (ODT) Containing Guanfacine, Un-Micronized Resin-Guanfacine Complexes, and Micronized Resin-Guanfacine Complexes

Orally Disintegrating tablets (ODT) containing Guanfacine HCl active, un-micronized resin-Guanfacine complexes, and micronized resin-Guanfacine complexes were prepared by compressing blends prepared in Examples 11, 12, 13 respectively, using the following tablet press and punches.

Tablet Press: GlobePharma Model MP-2B10 with 10 stations.

Punches: 7.94 mm diameter, round shape, Type B

Tablet properties (n=10) are summarized below.

| | | ODT Tablets | ODT Tablets |
|---|---|---|---|
| | | Comprising | Comprising |
| | | Un-micronized | Micronized |
| | ODT Tablets | resin- | resin- |
| | Comprising | Guanfacine | Guanfacine |
| | Guanfacine | complex | complex |
| Product Name | (9055-35) | (9055-37) | (9055-39) |
| Average Tablet Weight | 195.4 ± 1.1 | 198 ± 1.1 | 202.1 ± 1.0 |
| (mg) ± RSD % | | | |
| Average Tablet Thickness | 4.11 ± 1.3 | 4.27 ± 0.8 | 4.31 ± 1.2 |
| (mm) ± RSD% | | | |

### Example 15 Preparation of Orally Disintegrating Mini-Tablets Containing Guanfacine HCL, Un-Micronized Resin-Guanfacine Complexes, and Micronized Resin-Guanfacine Complexes

Mini-tablets containing Guanfacine HCl active, un-micronized resin-Guanfacine complexes, and micronized resin-Guanfacine complexes were prepared by compressing blends prepared in Examples 11, 12, 13 respectively, using the following tablet press and punches.

Tablet Press: GlobePharma Model MP-2B10 with 10 stations.

Punches: 2 mm diameter pin, round shape, 14 tips per punch, Type B

Tablet properties (n=10) are summarized below.

| | | Mini tablets | Mini tablets |
|---|---|---|---|
| | | Comprising | Comprising |
| | | Un-micronized | Micronized |
| | Mini tablets | resin- | resin- |
| | Comprising | Guanfacine | Guanfacine |
| | Guanfacine | complex | complex |
| Product Name | (9055-41B) | (9055-43) | (9055-45) |
| Tablet Weight | 6.067 ± 2.8 | 5.974 ± 2.1 | 5.967 ± 2.3 |
| (mg) ± RSD % | | | |
| Tablet Thickness | 2.551 ± 0.9 | 2.597 ± 1.1 | 2.581 ± 1.2 |
| (mm) ± RSD % | | | |

### Example 16: Demonstrate the Benefits of Micronized Resin-Therapeutic Agent Complexes on Active Uniformity in Orally Disintegrating Tablet (ODT)

The ODT tablets prepared in Example 14 that contain pure active Guanfacine HCl, un-micronized ion-exchange resin-guanfacine complexes, and micronized ion-exchange resin-guanfacine complexes were tested for drug content uniformity. The drug content was determined for ten tablets and the mean and relative standard deviation (RSD %) are calculated and compared. The results are summarized in the table below.

Drug Content of ODT tablets comprising Guanfacine HCl active only (without ion exchange resin)

| | Tablet | | | Guanfacine | | |
|---|---|---|---|---|---|---|
| | Wt | Mean | | Content | Mean | |
| Sample # | (mg) | (mg) | RSD | (mg) | (mg) | RSD |
| 9055-35-CU-1 | 197.5 | 197.7 | 0.36% | 2.0 | 2.1 | 1.05% |
| 9055-35-CU-2 | 198.4 | | | 2.1 | | |
| 9055-35-CU-3 | 198.5 | | | 2.1 | | |
| 9055-35-CU-4 | 197.9 | | | 2.1 | | |
| 9055-35-CU-5 | 196.7 | | | 2.1 | | |
| 9055-35-CU-6 | 197.6 | | | 2.1 | | |
| 9055-35-CU-7 | 197.6 | | | 2.1 | | |
| 9055-35-CU-8 | 198.6 | | | 2.1 | | |
| 9055-35-CU-9 | 196.5 | | | 2.1 | | |
| 9055-35-CU-10 | 197.5 | | | 2.1 | | |

Drug Content of ODT tablets comprising un-micronized resin-Guanfacine complexes

| | Tablet | | | Guanfacine | | |
|---|---|---|---|---|---|---|
| | Wt | Mean | | Content | Mean | |
| Sample # | (mg) | (mg) | RSD | (mg) | (mg) | RSD |
| 9055-37-CU-1 | 201.0 | 201.7 | 0.89% | 1.69 | 1.7 | 4.99% |
| 9055-37-CU-2 | 205.2 | | | 1.91 | | |
| 9055-37-CU-3 | 200.9 | | | 1.77 | | |
| 9055-37-CU-4 | 199.6 | | | 1.62 | | |
| 9055-37-CU-5 | 202.1 | | | 1.74 | | |
| 9055-37-CU-6 | 201.4 | | | 1.80 | | |
| 9055-37-CU-7 | 199.0 | | | 1.62 | | |
| 9055-37-CU-8 | 202.4 | | | 1.72 | | |
| 9055-37-CU-9 | 202.8 | | | 1.75 | | |
| 9055-37-CU-10 | 203.0 | | | 1.76 | | |

Drug Content of ODT tablets comprising micronized resin-Guanfacine complex

| | Tablet | | | Guanfacine | | |
|---|---|---|---|---|---|---|
| | Wt | Mean | Content | Mean | | |
| Sample # | (mg) | (mg) | RSD | (mg) | (mg) | RSD |
| 9055-39-CU-1 | 201.0 | 203.81 | 0.88% | 1.77 | 1.8 | 1.68% |
| 9055-39-CU-2 | 205.2 | | | 1.78 | | |
| 9055-39-CU-3 | 200.9 | | | 1.75 | | |
| 9055-39-CU-4 | 199.6 | | | 1.78 | | |
| 9055-39-CU-5 | 202.1 | | | 1.71 | | |
| 9055-39-CU-6 | 201.4 | | | 1.79 | | |
| 9055-39-CU-7 | 199.0 | | | 1.73 | | |
| 9055-39-CU-8 | 202.4 | | | 1.79 | | |
| 9055-39-CU-9 | 202.8 | | | 1.71 | | |
| 9055-39-CU-10 | 203.0 | | | 1.74 | | |

The data show that the tablet weight of these batches were well controlled to minimize their effect on drug content uniformity. The results demonstrated that the best uniformity in drug content of these ODT tablets was produced by granulating using dissolved guanfacine solution which resulted in 1.05% RSD. The worst uniformity in drug content of these ODT tablets was produced by using un-micronized resin-active complex which resulted in 4.99% RSD. The uniformity in drug content produced by using micronized resin-active complex resulted in a RSD (1.68%) closer to tablets produced by guanfacine solution granulation and much better than the tablets produced by un-micronized resin-active complex. Even though the tablets produced by guanfacine solution granulation give best drug content uniformity, these tablets bear strong bitter taste of guanfacine. Only tablets produced by using micronized resin-active complex provide good drug content uniformity and best taste & mouthfeel.

### Example 17: Demonstrate the Benefit of Resin-Therapeutic Agent Complexes on Active Uniformity in Mini-Tablets

The minitablets prepared in Example 15 that contains pure active Guanfacine HCl, un-micronized ion-exchange resin-guanfacine complexes, and micronized ion-exchange resin-guanfacine complexes were tested for drug content uniformity. The drug content was determined for ten tablets and the mean and relative standard deviation (RSD %) are calculated and compared. The results are summarized in the table below.

Drug Content of Mini tablets comprising Guanfacine HCl active only (without ion exchange resin)

| | | | | Guan- | | |
|---|---|---|---|---|---|---|
| | Tablet | | | facine | | |
| | Wt | Mean | | Content | Mean | |
| Sample # | (mg) | (mg) | RSD | (mg) | (mg) | RSD |
| 9055-41-B-CU-#1 | 6.3410 | 6.1486 | 2.13% | 0.064 | 0.064 | 3.75% |
| 9055-41-B-CU-#2 | 6.0470 | | | 0.064 | | |
| 9055-41-B-CU-#3 | 6.2110 | | | 0.064 | | |
| 9055-41-B-CU-#4 | 6.1250 | | | 0.064 | | |
| 9055-41-B-CU-#5 | 6.0850 | | | 0.069 | | |
| 9055-41-B-CU-#6 | 6.0350 | | | 0.067 | | |
| 9055-41-B-CU-#7 | 6.1740 | | | 0.066 | | |
| 9055-41-B-CU-#8 | 6.0230 | | | 0.061 | | |
| 9055-41-B-CU-#9 | 6.0520 | | | 0.063 | | |
| 9055-41-B-CU-#10 | 6.3930 | | | 0.062 | | |

Drug Content of Mini tablets comprising un-micronized resin-Guanfacine complex

| | Tablet | | | Guanfacine | | |
|---|---|---|---|---|---|---|
| | Wt | Mean | | Content | Mean | |
| Sample # | (mg) | (mg) | RSD | (mg) | (mg) | RSD |
| 9055-43-CU-#1 | 6.0610 | 6.0594 | 1.51% | 0.054 | 0.055 | 5.24% |
| 9055-43-CU-#2 | 6.0380 | | | 0.050 | | |
| 9055-43-CU-#3 | 6.1390 | | | 0.057 | | |
| 9055-43-CU-#4 | 6.1410 | | | 0.056 | | |
| 9055-43-CU-#5 | 6.1550 | | | 0.056 | | |
| 9055-43-CU-#6 | 6.1030 | | | 0.058 | | |
| 9055-43-CU-#7 | 6.1400 | | | 0.058 | | |
| 9055-43-CU-#8 | 5.9570 | | | 0.056 | | |
| 9055-43-CU-#9 | 5.9320 | | | 0.051 | | |
| 9055-43-CU-#10 | 5.9280 | | | 0.052 | | |

Drug Content of Mini tablets comprising micronized resin-Guanfacine complex

| | Tablet | | | Guanfacine | | |
|---|---|---|---|---|---|---|
| | Wt | Mean | | Content | Mean | |
| Sample # | (mg) | (mg) | RSD | (mg) | (mg) | RSD |
| 9055-45-CU-#1 | 6.1420 | 6.0434 | 1.37% | 0.056 | 0.055 | 3.82% |
| 9055-45-CU-#2 | 5.9680 | | | 0.054 | | |
| 9055-45-CU-#3 | 6.1330 | | | 0.057 | | |
| 9055-45-CU-#4 | 5.9340 | | | 0.052 | | |
| 9055-45-CU-#5 | 5.9930 | | | 0.055 | | |
| 9055-45-CU-#6 | 6.0450 | | | 0.054 | | |
| 9055-45-CU-#7 | 6.1480 | | | 0.056 | | |
| 9055-45-CU-#8 | 6.1010 | | | 0.059 | | |
| 9055-45-CU-#9 | 6.0230 | | | 0.054 | | |
| 9055-45-CU-#10 | 5.9470 | | | 0.052 | | |

The data show that the tablet weights of these batches were well controlled to minimize their effect on drug content uniformity. The results demonstrated that mini tablets produced by using micronized resin-Guanfacine complex possess a similar drug content uniformity as the mini tablets produced by granulating using guanfacine HCl solution. The mini tablets produced by using micronized resin-Guanfacine complex will have additional benefits of concealing guanfacine bitter taste and better mouthfeel of the micronized complex particles. In contract, the mini tablets produced by using un-micronized resin-guanfacine complex showed higher variation in drug content uniformity. The results demonstrated the benefit of micronized resin-Guanfacine complex in improving content uniformity in challenging pharmaceutical dosage form such as mini tablets which meet special needs for patients such as young children.

What is claimed is:

1. A pharmaceutical composition for oral administration, comprising:
micronized ion-exchange resin particles having particle sizes less than 50 μm, and
at least one therapeutic agent releasably bound to the micronized resin particles through ionic interaction to form resin-therapeutic agent complexes,
wherein the resin-therapeutic agent complexes have particle sizes less than 50 μm, and
wherein the pharmaceutical composition is formulated as a dosage form being either as a liquid dosage form or a solid dosage form, where the liquid dosage form has uniform dispersion of the resin-therapeutic agent complexes with less than 1 wt % of the resin particles in the form of aggregates, and wherein the solid dosage form provides uniform dispersion of the resin-therapeutic agent complexes with less than 1 wt % of the micronized resin particles in the form of aggregates when re-dispersed/constituted in a liquid medium, and
thereby the taste of the therapeutic agent is substantially masked and the dosage form has a reduced gritty mouth feel compared to a dosage form containing resin particles with particle sizes larger than 50 μm.

2. The pharmaceutical composition of claim 1, wherein in the solid dosage further comprises a dispersing agent, thereby when the pharmaceutical composition is re-dispersed in a liquid medium, uniform dispersion of the resin-therapeutic agent complexes is produced and less than 1 wt % of the resin particles are in the form of aggregates.

3. The pharmaceutical composition of claim 2, wherein the dispersing agent is:
a water-soluble substance selected from the group consisting of water-soluble polymers, hydrophilic surfactants, sugars, such as gelatin, Arabic gum, sodium alginate, agarose, polyvinyl pyrrolidone, hydroxypropyl methylcellulose, hydroxypropyl cellulose, sodium carboxymethyl cellulose, mannitol, lactose, sucrose, sodium lauryl sulfate, poloxamers, or combinations thereof, or
a solvent-soluble material selected from the group consisting of solvent-soluble polymers, lipophilic surfactants, phospholipids, fatty acids, such as polyvinyl pyrrolidone, phosphatidylcholine, phosphatidylethanolamine, stearate acid, oleic acid, or combinations thereof.

4. The pharmaceutical composition of claim 1, wherein the dosage form is selected from the group consisting of suspension, dry powder for suspension, orally disintegrating tablets, mini-tablets with the longest dimension less than or equal to 3 mm, chewable tablets, buccal tablets, oral films, oral jelly, and oral gummies.

5. The pharmaceutical composition of claim 4, wherein the dosage form is a liquid suspension comprising:
(a) between about 0.4% (w/w) and about 50% (w/w) of the therapeutic agent;
(b) between about 0.4% (w/w) and about 84.6% (w/w) of the micronized ion-exchange particles;
(c) between about 0% (w/w) and about 60% (w/w) of a dispersing aid or a dispersing agent;
(d) between about 0% (w/w) and about 40% (w/w) of a suspending agent or thickener;
(e) between about 0% (w/w) and about 20% (w/w) of an additional agent selected from the group consisting of a flavoring agent, a preservative, a pH adjusting agent, an antifoaming agent, a coloring agent, an antioxidant and a combination thereof; and
(f) between about 15% (w/w) and about 80% (w/w) of purified water.

6. The pharmaceutical composition of claim 1, wherein the particle size of the resin-therapeutic agent complex is from about 0.5 μm to about 40 μm.

7. The pharmaceutical composition of claim 1, wherein the particle size of the resin-therapeutic agent complex is from about 1 μm to about 30 μm.

8. The pharmaceutical composition of claim 1, wherein the particle size of the resin-therapeutic agent complex is from about 1 μm to about 20 μm.

9. The pharmaceutical composition of claim 1, wherein the ratio of the therapeutic agent to the resin particle is in the range from 5:1 to 1:100 by weight, calculated on a moisture-free basis.

10. The pharmaceutical composition of claim 9, wherein the ratio of the therapeutic agent to the resin particle is in the range from 2:1 to 1:20 by weight.

11. The pharmaceutical composition of claim 1, wherein the resin particle is an anionic ion-exchange resin particle.

12. The pharmaceutical composition of claim 1, wherein the resin particle is a cationic ion-exchange resin particle.

13. The pharmaceutical composition of claim 1, wherein the resin particle has an ion-exchange capacity of less than 6 milliequivalents per gram (meq/g) of dry resin.

14. The pharmaceutical composition of claim 1, wherein the resin particle is a cross-linked sulfonated polystyrene ion-exchange resin, a cross-linked methacrylic acid and divinylbenzene copolymer ion-exchange resin, a cross-linked copolymer of diethylenetriamine and 1-chloro-2,3-epoxy propane ion-exchange resin, or a cross-linked copolymer of styrene and divinylbenzene with quaternary ammonium functionality ion-exchange resin

15. The pharmaceutical composition of claim 1, wherein the resin particle is Amberlite IRP-69, Amberlite IRP-64, Colestipol hydrochloride, or Duolite AP143/1093

16. The pharmaceutical composition of claim 1, wherein the pharmaceutical composition comprises from about 1% to about 100% by weight of the drug-resin particles.

17. The pharmaceutical composition of claim 1, wherein the therapeutic agent is acidic.

18. The pharmaceutical composition of claim 17, wherein the therapeutic agent contains a carboxyl group.

19. The pharmaceutical composition of claim 17, wherein the therapeutic agent is selected from the group consisting of dehydrocholic acid, diflunisal, ethacrynic acid, fenoprofen, furosemide, gemfibrozil, ibuprofen, naproxen, phenytoin, probenecid, sulindac, theophylline, salicylic acid, and acetylsalicylic acid.

20. The pharmaceutical composition of claim 1, wherein the therapeutic agent is basic.

21. The pharmaceutical composition of claim 20, wherein the therapeutic agent contains an amine group.

22. The pharmaceutical composition of claim 20, wherein the therapeutic agent is selected from the group consisting of acetophenazine, amitriptyline, amphetamine, benztropine, biperiden, bromodiphenhydramine, brompheniramine, carbinoxamine, chlorcyclizine, chlorpheniramine, chlorphenoxamine, chlorpromazine, clemastine, clomiphene, clonidine, codeine, cyclizine, cyclobenzaprine, cyproheptadine, desipramine, dexbrompheniramine, dexchlorpheniramine, dextroamphetamine, dextromethorphan, dicyclomine, diphemanil, diphenhydramine, doxepin, doxylamine, ergotamine, fluphenazine, haloperidol, hydrocodone, hydroxychloroquine, hydroxyzine, hyoscyamine, imipramine, levopropoxyphene, maprotiline, meclizine, mepenzolate, meperidine, mephentermine, mesoridazine, methadone, methdilazine, methscopolamine, methysergide, metoprolol, nortriptyline, noscapine, nylindrin, orphenadrine, papaverine, pentazocine, phendimetrazine, phentermine, phenylpropanolamine, pyrilamine, tripelennamine, triprolidine, promazine, propoxyphene, propanolol, pseudoephedrine, pyrilamine, quinidine, scopolamine, dextromethorphan, chlorpheniramine, and codeine.

23. The pharmaceutical composition of claim 1, wherein the therapeutic agent is amphoteric.

24. The pharmaceutical composition of claim 23, wherein the therapeutic agent is selected from the group consisting of aminocaproic acid, aminosalicylic acid, hydromorphone, isoxsuprine, levorphanol, melphalan, morphine, nalidixic acid, and para-aminosalicylic acid.

25. The pharmaceutical composition of claim 1, wherein the therapeutic agent is selected from the group consisting of analeptic agents; analgesic agents; anesthetic agents; antiasthmatic agents; antiarthritic agents; anticancer agents; anticholinergic agents; anticonvulsant agents; antidepressant agents, antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism active agents; antipruritic agents; antipsychotic agents; antipyretic agents; antispasmodic agents; antitubercular agents; antiulcer agents; antiviral agents; anxiolytic agents; appetite Suppressants (anorexic agents); attention deficit disorder and attention deficit hyper activity disorder active agents; cardiovascular agents including calcium channel blockers and antianginal agents; central nervous system (CNS) agents; beta-blockers and antiar rhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasym patholytics; peptide active agents; psychoStimulants; sedatives; Sialagogues, steroids; Smoking cessation agents; Sympathomimetics; tranquilizers; vasodilators; beta-agonist; tocolytic agents; and combinations thereof.

26. The pharmaceutical composition of claim 1, wherein the therapeutic agent is an anti-cancer or anti-tumor agent, an anti-viral, or an anti-bacterial agent.

27. A method for preparing a pharmaceutical composition for oral administration, comprising:
micronizing ion-exchange resin particles by subjecting a suspension comprising ion-exchange resin particles having particle sizes larger than 50 μm to a size reduction process one or more times to obtain micronized ion-exchange resin particles having particle sizes equal to or less than 50 μm;
contacting the resulting micronized ion-exchange resin particles with at least one therapeutic agent to form resin-therapeutic agent complexes; and
admixing with a pharmaceutically acceptable carrier to form either a liquid dosage form having uniform dispersion of the resin-therapeutic agent complexes with less than 1 wt % of the resin particles in the form of aggregates, or a solid dosage form that, when re-dispersed/constituted in a liquid medium, provides uniform dispersion of the resin-therapeutic agent complexes with less than 1 wt % of the micronized resin particles in the form of aggregates.

28. A method for preparing a pharmaceutical composition for oral administration, comprising:
contacting ion-exchange resin particles having particle sizes larger than 50 μm with at least one therapeutic agent to form resin-therapeutic agent complexes;
micronizing the resin-therapeutic agent complexes by subjecting the resin-therapeutic agent complexes to a size reduction process one or more times to obtain micronized resin-therapeutic agent complexes having particle sizes equal to or less than 50 μm; and
admixing with a pharmaceutically acceptable carrier to yield a liquid dosage form having uniform dispersion of the resin-therapeutic agent complexes with less than 1 wt % of the resin particles in the form of aggregates, or a solid dosage form that, when re-dispersed/constituted in a liquid medium, provides uniform dispersion of the resin-therapeutic agent complexes with less than 1 wt % of the micronized resin particles in the form of aggregates.

29. The method of claim 27, wherein the size reduction process is jet milling, media milling, microfluidizer milling, or high-pressure homogenization.

30. The method of claim 27, wherein the ion-exchange resin particles and the therapeutic agent are provided at a ratio of the therapeutic agent to the resin particle is in the range from 5:1 to 1:100 by weight, calculated on a moisture-free basis.

31. The method of claim 30, wherein the ratio of the therapeutic agent to the resin particle is in the range from 2:1 to 1:20 by weight.

32. The method of claim 27, further comprising adding a dispersing agent before or after the step of micronizing, thereby when the pharmaceutical composition is re-dispersed in a liquid medium, uniform dispersion of the resin-therapeutic agent complexes is produced and less than 1 wt % of the resin particles are in the form of aggregates.

33. The method of claim 32, wherein the dispersing agent is:
a water-soluble substance selected from the group consisting of water-soluble polymers, hydrophilic surfactants, sugars, such as gelatin, Arabic gum, sodium alginate, agarose, polyvinyl pyrrolidone, hydroxypropyl methylcellulose, hydroxypropyl cellulose, sodium carboxymethyl cellulose, mannitol, lactose, sucrose, sodium lauryl sulfate, poloxamers, Tweens, or combinations thereof, or
a solvent-soluble material selected from the group consisting of solvent-soluble polymers, lipophilic surfactants, phospholipids, fatty acids, such as polyvinyl pyrrolidone, phosphatidylcholine, phosphatidylethanolamine, stearate acid, oleic acid, Spans, or combinations thereof.

34. The method of claim 27, comprising formulating the pharmaceutical composition as a dosage form selected from the group consisting of suspension, orally disintegrating tablets, mini tablets with the longest dimension less than or equal to 3 mm, chewable tablets, oral jelly, and oral gummies.

35. The method of claim 34, wherein the dosage form is a liquid suspension comprising:
(a) between about 0.4% (w/w) and about 50% (w/w) of the therapeutic agent;
(b) between about 0.4% (w/w) and about 84.6% (w/w) of the micronized ion-exchange particles;
(c) between about 0% (w/w) and about 60% (w/w) of a dispersing aid or a dispersing agent;
(d) between about 0% (w/w) and about 40% (w/w) of a suspending agent or thickener;
(e) between about 0% (w/w) and about 20% (w/w) of an additional agent selected from the group consisting of a flavoring agent, a preservative, a pH adjusting agent, an antifoaming agent, a coloring agent, an antioxidant and a combination thereof; and
(f) between about 15% (w/w) and about 80% (w/w) of purified water.

36. The method of claim 34, wherein the dosage form is a dry powder for suspension comprising:
(a) between about 0.4% (w/w) and about 20% (w/w) of the therapeutic agent;
(b) between about 20% (w/w) and about 99.6% (w/w) of the micronized ion-exchange particles;
(c) between about 0% (w/w) and about 60% (w/w) of a dispersing aid or a dispersing agent;
(d) between about 0% (w/w) and about 40% (w/w) of a thickener; and
(e) between about 0% (w/w) and about 20% (w/w) of an additional agent selected from the group consisting of a flavoring agent, a preservative, a pH adjusting agent, an antifoaming agent, a coloring agent, an antioxidant and a combination thereof.

37. The method of claim 27, wherein the particle size of the resin-therapeutic agent complex is from about 1 μm to about 40 μm.

38. The method of claim 37, wherein the particle size of the resin-therapeutic agent complex is from about 1 μm to about 30 μm.

39. The method of claim 37, wherein the particle size of the resin-therapeutic agent complex is from about 1 μm to about 20 μm.

40. The method of claim 27, wherein the resin particle is an anionic ion-exchange resin particle.

41. The method of claim 27, wherein the resin particle is a cationic ion-exchange resin particle.

42. The method of claim 27, wherein the resin particle has an ion-exchange capacity of less than 6 milliequivalents per gram (meq/g) of dry resin.

43. The method of claim 27, wherein the resin particle is a cross-linked sulfonated polystyrene ion-exchange resin, a cross-linked methacrylic acid and divinylbenzene copolymer ion-exchange resin, a cross-linked copolymer of diethylenetriamine and 1-chloro-2,3-epoxy propane ion-exchange resin, or a cross-linked copolymer of styrene and divinylbenzene with quaternary ammonium functionality ion-exchange resin

44. The method of claim 27, wherein the resin particle is Amberlite IRP-69, Amberlite IRP-64, Colestipol hydrochloride, or Duolite AP143/1093

45. The method of claim 27, wherein the pharmaceutical composition comprises from about 1 percent to about 50 percent by weight of the drug-resin particles.

46. The method of claim 27, wherein the therapeutic agent is acidic.

47. The method of claim 46, wherein the therapeutic agent contains a carboxyl group.

48. The method of claim 47, wherein the therapeutic agent is selected from the group consisting of dehydrocholic acid, diflunisal, ethacrynic acid, fenoprofen, furosemide, gemfibrozil, ibuprofen, naproxen, phenytoin, probenecid, sulindac, theophylline, salicylic acid, and acetylsalicylic acid.

49. The method of claim 27, wherein the therapeutic agent is basic.

50. The method of claim 49, wherein the therapeutic agent contains an amine group.

51. The method of claim 50, wherein the therapeutic agent is selected from the group consisting of acetophenazine, amitriptyline, amphetamine, benztropine, biperiden, bromodiphenhydramine, brompheniramine, carbinoxamine, chlorcyclizine, chlorpheniramine, chlorphenoxamine, chlorpromazine, clemastine, clomiphene, clonidine, codeine, cyclizine, cyclobenzaprine, cyproheptadine, desipramine, dexbrompheniramine, dexchlorpheniramine, dextroamphetamine, dextromethorphan, dicyclomine, diphemanil, diphenhydramine, doxepin, doxylamine, ergotamine, fluphenazine, haloperidol, hydrocodone, hydroxychloroquine, hydroxyzine, hyoscyamine, imipramine, levopropoxyphene, maprotiline, meclizine, mepenzolate, meperidine, mephentermine, mesoridazine, methadone, methdilazine, methscopolamine, methysergide, metoprolol, nortriptyline, noscapine, nylindrin, orphenadrine, papaverine, pentazocine, phendimetrazine, phentermine, phenylpropanolamine, pyrilamine, tripelennamine, triprolidine, promazine, propoxyphene, propranolol, pseudoephedrine, pyrilamine, quinidine, scopolamine, dextromethorphan, chlorpheniramine, and codeine.

52. The method of claim 27, wherein the therapeutic agent is amphoteric.

53. The method of claim 52, wherein the therapeutic agent is selected from the group consisting of aminocaproic acid, aminosalicylic acid, hydromorphone, isoxsuprine, levorphanol, melphalan, morphine, nalidixic acid, and para-aminosalicylic acid.

54. The method of claim 27, wherein the therapeutic agent is selected from the group consisting of analeptic agents; analgesic agents; anesthetic agents; antiasthmatic agents; antiarthritic agents; anticancer agents; anticholinergic agents; anticonvulsant agents; antidepressant agents, antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism active agents; antipruritic agents; antipsychotic agents; antipyretic agents; antispasmodic agents; antitubercular agents; antiulcer agents; antiviral agents; anxiolytic agents; appetite Suppressants (anorexic agents); attention deficit disorder and attention deficit hyper activity disorder active agents; cardiovascular agents including calcium channel blockers and antianginal agents; central nervous system (CNS) agents; beta-blockers and antiar rhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasym patholytics; peptide active agents; psychoStimulants; sedatives; Sialagogues, steroids; Smoking cessation agents; Sympathomimetics; tranquilizers; vasodilators; beta-agonist; tocolytic agents; and combinations thereof.

55. The method of claim 27, wherein the therapeutic agent is an anti-cancer or anti-tumor agent, an anti-viral, or an anti-bacterial agent.
